(19) United States
(12) Patent Application Publication
(10) Pub. No.: US 2026/0092308 A1
(43) Pub. Date: Apr. 2, 2026

(54) ANALYTE DETECTION AND QUANTIFICATION

(71) Applicant: Early Is Good, Inc., Indianapolis, IN (US)

(72) Inventors: Thakshila Udayakanthi Habarakada Liyanage, Indianapolis, IN (US); Asel Ananda Habarakada Liyanage, Indianapolis, IN (US)

(21) Appl. No.: 19/110,396

(22) PCT Filed: Sep. 15, 2023
(86) PCT No.: PCT/US2023/074376

§ 371 Date: Mar. 10, 2025

Related U.S. Application Data
(60) Provisional application No. 63/407,417, filed on Sep. 16, 2022.
(60) Provisional application No. 63/448,134, filed on Feb. 24, 2023.

(51) Int. Cl. C12Q 1/682; C12Q 1/66; C12Q 1/6806; C12Q 1/6825; G01N 21/64; G01N 21/65; G01N 21/76; G01N 33/543

(52) U.S. Cl. CPC C12Q 1/682; C12Q 1/66; C12Q 1/6806; C12Q 1/6825; G01N 21/648; G01N 21/658; G01N 21/763; G01N 33/54373; C12Q 2600/178; G01N 2333/90241

(57) ABSTRACT

The present features methods and components for analyte detection in general, methods and components for quantifying analytes associated with bladder cancer, and treatment of patients identified as having analyte levels predictive of bladder cancer. The general methods and components can be used to detect the presence of small amounts of analytes in sample.

### CROSS REFERENCE TO RELATED APPLICATION

The present application claims priority to U.S. Provisional Application No. 63/407,417 filed on Sep. 16, 2022, and U.S. Provisional Application No. 63/448,134, filed Feb. 24, 2023, the disclosures of which is by reference incorporated herein in their entirety.

### REFERENCE TO SEQUENCE LISTING SUBMITTED ELECTRONICALLY

The contents of the electronic sequence listing (068469_P1WO1.xml; Size: 27,355 bytes; and Date of Creation: Sep. 6, 2023) is herein incorporated by reference in its entirety.

### FIELD OF THE INVENTION

The present invention relates to different fields, including methods and components for analyte detection and quantification in general, methods and components for detecting analytes associated with bladder cancer, and bladder cancer treatment.

### BACKGROUND OF THE INVENTION

Plasmonic sensors can be used to detect the presence or amount of a variety of different analytes including environmental toxins, polysaccharides, bacteria, viruses, protein, enzymes, ligands, nucleic acid, substrates, exosomes, cells, drug levels, and tissues. Detection of a particular analyte is facilitated through the use of a capture molecule that specifically binds to the analyte, facilitating separation of the target analyte from other analytes. A variety of different plasmonic phenomena can be used to measure captured analytes including surface plasmon resonance, localized surface plasmon resonance, surface-enhanced Raman scattering, surface enhanced infrared absorption spectroscopy and surface-enhanced fluorescence.

Analyte detection and quantification is useful in many areas including detecting food contaminants and pathogens, detecting environmental toxins, and detecting biomarkers. Therapeutic and diagnostic applications of surface plasmon resonance based analyte detection include detecting biomarkers associated with a particular disease or order.

Biomarker detection and quantification can be used to identify subjects who would benefit from therapeutic intervention and to guide treatment. For example, surface plasmon resonance based analyte detection can be used in cancer detection and treatment. Clinical diagnosis is very important in early detection, monitoring tumor progression, and therapeutic response.

Bellassia et al., 2019 Front. Chem. 7:570 mentions the use of localized surface plasmon resonance and single-stranded DNA, for detecting low levels of miR-182, miR-10b, miR-143 and miR-145 associated with bladder cancer.

Habarakada Liyanage et al., US Patent Publication No. 2021/0140911 includes descriptions of a system and method for lateral flow technology that can be used to detect analytes, localized surface plasmon resonance, and different biomarkers including biomarkers associated with bladder cancer.

### BRIEF SUMMARY OF THE INVENTION

The present features methods and components for analyte detection and quantification in general, methods and components for quantifying analytes associated with bladder cancer, and treatment of patients identified as having analyte levels predictive of bladder cancer. The general methods and components are illustrated with respect to quantification of different analytes associated with bladder cancer. The provided combination of bladder analytes can also be detected using other methods.

Thus, a first aspect of the present invention relates to an analyte detection signal amplifier. The amplifier comprises (a) an analyte binding molecule; (b) a localized surface plasmon resonance nanostructure; and (c) either (i) a bioluminescence resonance energy transfer (BRET) assembly complex, wherein the BRET assembly complex comprises a luciferase donor conjugated to a fluorophore acceptor or (ii) a fluorescence resonance energy transfer (FRET) assembly complex, wherein the FRET assembly complex comprises a fluorescent donor conjugated to a fluorophore acceptor.

Another aspect of the present invention relates to detecting or quantifying an analyte in a sample comprising the steps of:
a) contacting a localized surface plasmon resonance sensor with the sample, wherein the sensor comprises an analyte capture molecule;
b) providing an analyte detection signal amplifier to the sample, wherein the analyte detection signal amplifier comprises a BRET assembly complex and a localized surface plasmon resonance nanostructure and binds to the analyte;
c) adding a luciferase substrate; and
d) detecting fluorescence, bioluminescence, localized surface plasmon resonance or surface-enhanced Raman scattering.

Another aspect of the present invention relates to detecting or quantifying an analyte in a sample comprising the steps of:
a) contacting a localized surface plasmon resonance sensor with the sample, wherein the sensor comprises an analyte capture molecule;
b) providing the analyte detection signal amplifier to the sample, wherein the analyte detection signal amplifier comprises a FRET assembly complex and a localized surface plasmon resonance nanostructure and binds to the analyte;
c) excitation; and
d) detecting fluorescence, localized surface plasmon resonance, or surface-enhanced Raman scattering.

Another aspect of the present invention relates to a multi-analyte detection system for detecting analytes whose presence or amount is associated with bladder cancer. The system comprises:
a) a first localized surface plasmon nanostructure sensor comprising an analyte capture molecule for miR-205;
b) a second localized surface plasmon nanostructure sensor comprising an analyte capture molecule for miR-16-1;
c) a third localized surface plasmon nanostructure sensor comprising an analyte capture molecule for miR-143;
d) a fourth localized surface plasmon nanostructure sensor comprising an analyte capture molecule for UCA1 nucleic acid;
e) a fifth localized surface plasmon nanostructure sensor comprising an analyte capture molecule for IGF2 or IGF2 nucleic acid; and
f) a sixth localized surface plasmon nanostructure sensor comprising an analyte capture molecule for ANXA10 or ANXA10 nucleic acid; wherein each of the sensor types may be present on one or more platforms.

Reference to each of the sensor types may be present on “one or more platforms” indicates that each platform may have a particular sensor (e.g., multiply sensors of the same type), or a particular platform may have two or more different types of sensors. In different embodiments, a particular platform has 1, 2, 3, 4, 5, 6, 7, 8, 9, 10, 11 or greater than 11 different types of sensors.

The BRET or FRET assembly complex and localized surface plasmon resonance nanostructures used in different signal amplifier types (e.g., the first, second, third, fourth, fifth or sixth) may be the same or different; and the localized surface plasmon resonance nanostructures used in different sensor types may be the same or different. The use of localized surface plasmon nanostructures having different compositions, different sizes and/or different shapes along with different BRET or FRET assembly complexes can be used to facilitate using different types of sensors and/or amplifiers on the same platform. In certain embodiments, each platform has the same or no more than three different types of sensors; each BRET or FRET are the same; each amplifier localized surface plasmon nanostructure is the same; and each sensor localized surface plasmon nanostructure is the same.

Another aspect of the present invention is directed to a method of determining whether a subject has bladder cancer comprising the steps of detecting the presence or amount of miR-205, miR-16-1, miR-143, UCA1 nucleic acid, IGF2 or IGF2 nucleic acid, and ANXA10 or ANXA10 nucleic acid.

Another aspect of the present invention is directed to a method of treating a subject for bladder cancer comprising administering a therapeutically effective amount of a bladder cancer therapeutic to a subject determined to have bladder cancer using the methods described herein.

Other features and advantages of the present invention are apparent from additional descriptions provided herein, including different examples. The provided examples illustrate different components and methodology useful in practicing the present invention. Such examples do not limit the claimed invention. Based on the present disclosure, the skilled artisan can identify and employ other components and methodology useful for practicing the present invention.

### BRIEF DESCRIPTION OF THE DRAWINGS

FIG. 1 illustrates a BRET assembly complex, where the BRET assembly complex comprises a luciferase donor (Bioluminescence donor) conjugated to a fluorophore acceptor through a Halo tag protein. The Halo tag protein is covalently conjugated to both the luciferase donor and fluorophore acceptor.

FIG. 2 provides an example of an overall scheme illustrating different aspects and embodiments.

FIGS. 3A and 3B show calibration plots measuring luminescence and fluorescence for different analytes. FIG. 3A illustrates CRH, ANAX10, ABL1, IGF2 and UPKB1 luminescence. FIG. 3B illustrates CRH, ANAX10, ABL1, IGF2 and UPKB1 fluorescence.

FIGS. 4A and 4B show calibration plots measuring luminescence and fluorescence for different analytes. FIG. 4A illustrates miR-200c (200c), miR-16-1 (16-1), miR-205 (205), miR-143 (143) and long non-coding RNA (UCA) luminescence. FIG. 4B illustrates miR-200c (200c), miR-16-1 (16-1), miR-205 (205), miR-143 (143) and long non-coding RNA (UCA) fluorescence.

FIGS. 5A and 5B show calibration plots measuring luminescence and fluorescence for different analytes. FIG. 5A illustrates NMP22, HCFHrp (hcfHRP) and P53 luminescence.

FIG. 5B illustrates NMP22, HCFHrp (hcfHRP) and P53 fluorescence.

### DETAILED DESCRIPTION OF THE INVENTION

The present features methods and components for analyte detection and quantification, methods and components for quantifying analytes associated with bladder cancer, and treatment of patients identified as having analyte levels predictive of bladder cancer. As illustrated in the examples provided below, the provided methods and components can quantitatively detect small amounts of analytes associated with bladder cancer. Analytes associated with bladder cancer include analytes where an increased level characterizes bladder cancer and analytes where a decreased level characterizes bladder cancer.

In certain embodiments, the method detects analytes present at a concentration as low as about 0.01 aM, 0.1 aM, or 10 aM. In further embodiments the detected analytes are present at a concentration below about 50 aM, 25 aM, 10 aM, 1 aM, or 0.1 aM.

Different subject areas involving analyte detection include food safety, for example, detecting contaminants such as pathogens, mycotoxins, plant and bio-marine toxins, toxic chemical, preservatives, and anti-oxidants; environment contaminants, such as pesticides, herbicides, aromatic compounds, chemical mixtures, and toxic metal contaminants; human security, for example, explosive and chemical warfare detection; and health areas, for example detecting biomarkers associated with a particular disease or disorder, including certain polypeptides, proteins, DNA, and RNA and detecting infectious organisms. (See, for example, Camarca et al., Sensors (Basel) 2021 Jan. 29; 21 (3): 906.)

Therapeutic applications of detecting and quantifying biomarkers associated with a particular disease and order can include diagnostic, prognostic, and treatment guiding. Diagnostic applications include early detection, and obtaining information on a particular type of a disease or disorder and the severity or stage of a disease or disorder. Prognostic applications include prediction of disease outcome and treatment response. Treatment guiding relates to the type of treatment selected, monitoring treatment, and adjusting treatment based on particular analyte levels.

Reference to “subject” indicates a mammal, including a human; non-human primates such as apes, gibbons, gorillas, chimpanzees, orangutans, and macaques; domestic animals such as dogs and cats; farm animals such as poultry, ducks, horses, cows, goats, sheep and pigs; and experimental animals such as mice, rats, rabbits, and guinea pigs. A preferred subject is a human.

The terms “nucleic acid” and “polynucleotide” are used interchangeably herein to refer to a nucleotide polymer without regard to function or size. Nucleic acid and polynucleotides contain at least two nucleotides and include deoxyribonucleic acid (DNA) and ribonucleic acid (RNA); and in some cases DNA or RNA derivatives having nitrogenous bases able to hydrogen bond to DNA or RNA, where the derivative contains a modified nucleobase, sugar moiety and/or phosphodiester linkage. In discussing nucleic acids and polynucleotides, unless otherwise indicated, a provided sequence is in the 5′ to 3′ direction.

The singular forms “a,” “an,” and “the” include plural reference unless the context clearly dictates otherwise.

The conjunctive term “and/or” between multiple recited elements encompasses both individual and combined options. For instance, where two elements are conjoined by “and/or”, a first option refers to the applicability of the first option without the second, a second option refers to the applicability of the second option without the first, and a third option refers to the applicability of the first and second options together. Any one of the options is understood to fall within the meaning and therefore satisfy the requirement of the term “and/or”. Concurrent applicability of more than one of the options is also understood to fall within the meaning of the term “and/or.”

Unless clearly indicated otherwise by the context employed the terms “or” and “and” have the same meaning as “and/or”.

Reference to terms such as “including”, “for example”, “e.g.,”, “such as” followed by different members or examples, are open-ended descriptions where the listed members or examples are illustrative and other members or examples can be provided or used.

The terms “polypeptide,” “protein” and “peptide” can be used interchangeably to refer to an amino acid sequence without regard to function. Polypeptides and peptides contain at least two amino acids, while proteins contain at least about 10 amino acids. The provided amino acids include naturally occurring amino acids and amino acids provided by cellular modification.

Reference to “comprise”, and variations such as “comprises” and “comprising”, used with respect to an element or group of elements is open-ended and does not exclude additional unrecited elements or method steps. Terms such as “including”, “containing” and “characterized by” are synonymous with comprising. In the different aspects and embodiments described herein reference to an open-ended term such as “comprising” can be replaced by “consisting” or “consisting essentially of”.

Reference to “consisting of” excludes any element, step, or ingredient not specified in the listed claim elements, where such element, step or ingredient is related to the claimed invention.

Reference to “consisting essentially of” limits the scope of a claim to the specified materials or steps and those that do not materially affect the basic and novel characteristic(s) of the claimed invention.

The term “about” refers to a value within 10% of the underlying parameter (i.e., plus or minus 10%). For example, “about 1:10” includes 1.1:10.1 or 0.9:9.9, and “about 5 hours” includes 4.5 hours or 5.5 hours. The term “about” at the beginning of a string of values modifies each of the values. In an embodiment, the term about refers to a range within 5% of the underlying parameter.

All numerical values or numerical ranges include integers within such ranges and fractions of the values or the integers within ranges unless the context clearly indicates otherwise. Reference to an integer with more (greater) or less than includes numbers greater or less than the reference number, respectively.

Various references including articles and patent publications are cited or described in the background and throughout the specification. Each of these references is herein incorporated by reference in their entirety. None of the references are admitted to be prior art with respect to any inventions disclosed or claimed. In some cases, particular references are indicated to be incorporated by reference herein to highlight the incorporation.

The definitions provided herein, including those in the present section and other sections of the application, apply throughout the present application.

Unless defined otherwise, all technical and scientific terms used herein have the same meaning commonly understood to one of ordinary skill in the art to which this invention pertains.

The description has been separated into various sections and paragraphs, and provides examples of various embodiments. These separations should not be considered as disconnecting the substance of a paragraph or section or embodiments from the substance of another paragraph or section or embodiment. The provided descriptions have broad application and encompass all the combinations of the various sections, paragraphs and sentences that can be contemplated. The discussion of any embodiment is meant only to be exemplary and is not intended to suggest the scope of the disclosure, including the claims (unless otherwise provided in the claims), is limited to these examples.

The instant invention is generally disclosed herein using affirmative language to describe the numerous embodiments of the instant invention. The instant invention also specifically includes embodiments in which particular subject matter is excluded, in full or in part, such as substances or materials, method steps and conditions, protocols, or procedures. For example, in certain embodiments of the instant invention, materials and/or method steps are excluded. Thus, even though the instant invention is generally not expressed herein in terms of what the instant invention does not include, embodiments that are not expressly excluded in the instant invention are nevertheless disclosed herein.

### I. Analyte Detection Signal Amplifier

An analyte detection signal amplifier comprises (a) an analyte binding molecule; and (b) a localized surface plasmon resonance nanostructure and either (i) a bioluminescence resonance energy transfer (BRET) assembly complex, or (ii) a fluorescence resonance energy transfer (FRET) assembly complex.

The analyte binding molecule specifically binds to an analyte. A variety of different types of analytes can be detected with appropriate binding molecules. Examples of different types of analytes that can be detected include protein, polypeptide, exosome, nucleic acid, virus, cell, tissue, polysaccharide, lipoprotein, lipid, and therapeutic compound.

In certain embodiments, the analyte is a protein or polypeptide. Examples of protein and polypeptide analytes that can be detected include enzymes, substrates, antibodies, protein hormones, receptors, and cytokines.

In certain embodiments, the analyte is a nucleic acid. Examples of nucleic acid that can be detected include RNA and DNA, such as mRNA, tRNA, siRNA, lncRNA, piwiRNA, snoRNA, tircRNA, mitochondrial RNA, non-coding RNA, miRNA, cDNA, genomic DNA, and enzymes (e.g., ribozymes).

In certain embodiments, the analyte is extracellular RNA, such as extracellular mRNA.

In certain embodiments, the analyte is an extracellular vesicle, such as extracellular exosomes. Extracellular exosomes can comprise different cellular components from the cells which they were derived, such as lipids, nucleic acid, and protein.

In certain embodiments, the analyte is a virus. Examples of viruses that can be detected include, pathogenic viruses infecting a subject, plant, agricultural product, or food; or present in the environment (for example water supply and ventilation).

In certain embodiments, the analyte is a cell. Examples of cells that can be detected include bacterial combinations (e.g., present in the gut); pathogenic bacteria infecting a subject, plant, agricultural product, or food; or present in the environment (for example water supply and ventilation).

In certain embodiments, the analyte is a polysaccharide. Examples of polysaccharides that can be detected include polysaccharides associated with bacteria or a particular disease or disorder in a subject.

In certain embodiments, the analyte is a lipoprotein. Examples of lipoprotein that can be detected include high density lipoprotein, low density, and bacterial lipoproteins.

In certain embodiments, the analyte is a lipid. Examples of lipids that can be detected include cholesterol and hormones.

In certain embodiments, the analyte is a therapeutic compound. Examples of therapeutic compounds that can be detected include small molecules, and large molecules such as antibodies and proteins.

In certain embodiments, the analyte is an environmental pollutant. Examples of pollutants that can be detected include air pollutants such as nitrogen oxides, and sulfur dioxide; water pollutants such as pesticides, heavy metals, and per- and polyfluoroalkyl substances (PFAS); and soil pollutants such as pesticides, heavy metals, and PFAS.

In certain embodiments, the analyte is an illegal drug.

Different configurations of an analyte binding molecule, localized surface plasmon resonance nanostructure, and the BRET assembly complex or (FRET) assembly complex are possible, such as: (1) the analyte binding molecule and the BRET or FRET assembly complexes are both linked to the localized surface plasmon resonance nanostructure; (2) the localized surface plasmon resonance nanostructure and BRET or FRET assembly are both linked to the antigen binding molecule; and (3) the localized surface plasmon resonance nanostructure and the analyte antigen binding molecule are both linked to the BRET or FRET assembly complex.

### I.A. Analyte Binding Molecule

Analyte binding molecules are able to specifically bind to an analyte of interest. Specific binding refers to the ability to bind to the analyte based upon the particular structure of the analyte, and distinguish (e.g., binds to a significantly greater extent) the targeted analyte from other analytes naturally present in a sample (e.g., biological sample). Absolute specificity while very helpful for some applications, may not be required. For example, the analyte binding molecule can be used in conjunction with a capture molecule, both of which are specific for the same analyte and assist in detecting the presence of the analyte. In different embodiments the binding molecule has a specificity to a target analyte at least 10×, at 100× or at least 1000× greater than other analytes present in the sample being tested.

In certain embodiments, the analyte binding molecule is a single-stranded binding oligonucleotide. A single-stranded binding oligonucleotide comprises purine or pyrimidine nucleobases or derivatives thereof, able to hydrogen bond via Watson-Crick hydrogen bonds with nucleobases present in DNA or RNA. Naturally occurring DNA and RNA contain a purine (guanine, cytosine, and the less common hypoxanthine) or a pyrimidine (thymine, uracil, or adenine) nucleobase, and a backbone made up of a ribose (RNA) or 2′-deoxyribose (DNA) joined together by phosphodiester groups.

Various modifications can be made to the different polynucleotide components to provide for modified oligonucleotides able to hydrogen bond to DNA or RNA having complementary nucleotide sequences. Examples of purine modifications include 2,6-diaminopurine, 3-deaza-adenine, 7-deasa-guanine, and 8-azido adenine. Examples of pyrimidine modifications include 2-thio-thymidine, 5-carboxamine-uracil, 5-methyl-cytosine, 3-ethynyl-uracil. Examples of phosphodiester modifications include methylphosphonate, phosphorothioate, and guanidinopropyl phosphoramidate. Examples of phosphate replacement includes triazole and guanidinium. Examples of sugar modifications include 2′-modifications such as 2′-F, 2′-methoxy, 2′-amino, and 2′-azido; locked sugar; 3′ end modifications; and 5′ end modification. (Ochoa and Milam, Molecules 2000, 25, 4689, hereby incorporated reference herein in its entirety.)

Another example of a modification includes peptide nucleic acid, where the sugar-phosphate backbone is replaced with a neutral pseudopeptide backbone. The peptide nucleic acid retains nucleobase complementarity, and the ability to hybridize to complementary DNA and RNA. Peptide nucleic acid can be produced, for example, by replacing the phosphodiester backbone with N-(2 aminoethyl) glycine, wherein the nucleobases are connected to the backbone via a methylene carbonyl linker. Additional peptide nucleic acid structures and design consideration are provided in Brodyagin et al., J. Org. Chem. 2021, 17, 1641-1688; and Moccia et al., Artif. DNA: PNA& XNA. 2014 5 (3): e1107176; each of which are hereby incorporated by reference herein in their entirety.

Oligonucleotide binding molecules can vary in size and degree of complementarity to target DNA or RNA. The degree of complementarity providing for specificity will vary depending oligonucleotide structure, for example, purine versus pyrimidine nucleobases, and modifications to the nucleobase, sugar and/or phosphodiester group; and the reaction conditions. In different embodiments concerning the binding oligonucleotide molecule size, the oligonucleotide comprises at least 10, at least 12, at least 15, at least 20, at least 30, at least 40, or at least 50, nucleobases. In different embodiments concerning the degree of complementarity to a target nucleic acid analyte, the oligonucleotide binding molecule comprises a region of 10 or more, 11 or more, 12 or more, 13 or more, 15 or more, 16 or more, 17 or more, 18 or more, 19 or more, 20 or more, 21 or more, 22 or more, 23 or more, 24 or more, or 25 or more nucleobases with at least 90%, at least 95% or 100% complementarity to a target nucleic acid analyte.

In certain embodiments the analyte binding molecule is an antibody or comprises a binding fragment of an antibody. An antibody binding fragment contains three complementary determining regions in a variable framework region allowing for antigen binding. Examples of binding fragments include FAb fragments, single chain variable region fragments (scFV), single domain fragments (dAbs), Fv fragment, camelid heavy-chain variable domains (VHHs), mini-body and diabody.

In certain embodiments the analyte binding molecule is an aptamer. Aptamers are small single-stranded oligonucleotides with a three-dimensional structure enabling specific binding to a target. The aptamer can be made up of naturally occurring nucleotides or can have one or more modifications. Aptamers can bind to a variety of targets including bacteria, viruses, proteins, toxins, cells (e.g., cancer cells) and tissues. Initial aptamer selection can be carried out using combinatorial oligonucleotide libraries through in vitro selection and iteration processes, such as Systematic Evolution of Ligands by Exponential Enrichment (SELEX). (Kulabhausan et al., Pharmaceutics 2020, 12, 646; and Adachi and Nakamura, Molecules 2019, 24, 4229; both of which are hereby incorporated by reference herein in their entirety.)

In certain embodiments the analyte binding molecule is a protein. Different types of protein can be used to bind to analytes, such an enzyme for binding to a substrate, a substrate for binding to an enzyme, a receptor for binding to a ligand, and a ligand for binding to a receptor.

In certain embodiments the analyte binding molecule is a ligand. Ligands may or may not be a protein.

The analyte binding molecule can be anchored directly to the localized surface plasmon resonance nanostructure or can be anchored through a linker. Depending on the localized surface plasmon resonance nanostructure, anchoring can be achieved through electrostatic interaction or covalent bonds.

Examples of nanoparticle surface immobilization chemistries include: (1) covalent coupling of nanoparticles with linker thiol groups; (2) covalent coupling of active ester-modified nanoparticles with linker amino groups; (3) coupling of maleimide-functionalized nanoparticles with thiol groups; (4) click reaction between dibenzocyclooctyne-modified nanoparticles and azido group linker; (5) electrostatic adsorption of negatively charged nanoparticles and positively charged linker group; and (6) biotin-NeutrAvidin-mediated linkages.

The length and composition of the analyte binding molecule linker can vary. The individual atoms of the linker can include atoms such as carbon, nitrogen, silicone, fluorine, oxygen, sulfur, hydrogen, and phosphorous.

The linker can comprise different types of groups or polymers such as polyethylene glycol (PEG), polyaminoacids, polyacrylamides, polyvinylpyrrolidon, zwitterionic polymers, polysaccharides, poly(N-(2-hydroxypropyl) methacrylamide), poly(oligo (ethylene glycol) methyl ether methacrylate), carboxylic dextran, hydrocarbon chains and substituted hydrocarbon chains.

In certain embodiments, the linker comprises PEG. Different types of PEG can be employed such as linear and branched of varying sizes. In certain embodiments the PEG is less than about 5K. The examples provided below illustrate PEG-SH (1K), having COOH and NH₂ functional groups.

In certain embodiments the PEG links directly to the analyte binding molecule where it acts as a linker (anchors to the nanoparticle) and spacer.

In certain embodiment the PEG is a spacer and is attached to the nanoparticle through another moiety. For example, the linker has a SH moiety attached directly to the nanoparticles and is immobilized onto nanoparticles via Au—S bond.

### I.B. BRET Assembly Complex

The BRET assembly complex comprises a luciferase donor joined to an acceptor fluorophore through a luciferase donor-acceptor linker. Following oxidation of a luciferase substrate, energy is transferred from the luciferase donor to excite the fluorophore acceptor. Emission from the luciferase donor and fluorophore can be measured. Emission can be characterized by, for example, wavelength, intensity, lifetime, and polarization.

Energy transfer between the luciferase donor and fluorophore acceptor occurs when the donor and acceptor are within approximately <10 nm of each other. The closer the donor and acceptor are to each other the stronger the emission.

In certain embodiments, the distance between luciferase donor and fluorophore acceptor is about 3 nm, about 4, nm, about 5 nm, about 6 nm, about 7 nm, about 8 nm, about 9 nm, or about 10 nm.

The luciferase donor-acceptor linker can be made up of atoms such as carbon, nitrogen, silicone, oxygen, sulfur, hydrogen, fluorine, and phosphorous,

The linker can comprise different types of groups or polymers such as PEG, polyaminoacids, polyacrylamides, polyvinylpyrrolidon, zwitterionic polymers, polysaccharides, poly(N-(2-hydroxypropyl) methacrylamide), poly(oligo (ethylene glycol) methyl ether methacrylate), carboxylic dextran, hydrocarbon chains and substituted hydrocarbon chains.

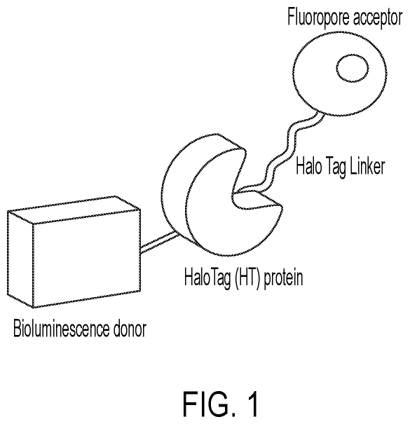

The linker provides a stable structure and can be made up of different groups joined together by different types of molecular interaction, and can be produced using different techniques. For example, the BRET assembly complex illustrated in FIG. 1 and the Examples below contains a luciferase molecule joined to a HaloTag protein, where the HaloTag protein is linked to a fluorophore molecule. The overall distance between the luciferase molecule and fluorophore, taking into the diameter of the haloTag protein joined to the luciferase donor (about 3.3 nm, see, e.g., Yazaki et al., Nucleic Acids Research, Volume 48, Issue 2, 24 Jan. 2020, Page e8) and the 12 atom linker (about 1.2 nm) to the fluorophore is about 4.5 nm Alternative techniques for joining different groups includes EDC/NHS coupling, click chemistry, streptavidin-biotin and complementary oligonucleotides.

Multiple variations of BRET have been developed using different luciferase donors, substates, and fluorophore acceptors. (See, e.g., Dale et al., Front. Bioeng. Biotechnol. 2019, 7, 56, hereby incorporated by reference herein in its entirety.) Examples of different variations are shown in Table 1.

TABLE 1

| Luciferase | | Donor | | Acceptor |
|---|---|---|---|---|
| Donor | | Emission | | emission |
| (Enzyme) | Substrate | (nm) | Fluorophore | (nm) |
| RLuc | Coelenterazine | 480 | eYFP | 530 |
| RLuc | Coelenterazine | 395 | GFP | 510 |
| | 400a (Deep | | | |
| | Blue C ™) | | | |
| RLuc8 | Coelenterazine | 395 | GFP | 510 |
| | 400a (Deep | | | |
| | Blue C ™) | | | |
| Firefly | Luciferin | 565 | DsRed | 583 |
| RLuc/RLuc8 | Coelenterazine | 480 | QDot | 605 |
| NanoLuc ®-HT | Furimazine | 460 | HaloTag ® | 618 |
| | | | Ligand | |
| NanoLuc ®-HT | Furimazine | 460 | HaloTag ® | 516 |
| | | | Ligand | |

GFP refers to green florescent protein, eYFP refers to enhanced yellow florescent protein, and RLuc refers to Renilla luciferase. NanoLuc®-HT is a nanoluciferase, also referred to as Nluc. Furimazine is 2-furanylmethyl-deoxy-coelenterazine.

Nluc was derived from the 19 kDa subunit of a larger multi-component luciferase isolated from the deep sea shrimp Oplophorus gracilirostris. The luminescence of the 19 kDa subunit was enhanced through mutagenesis and numerous coelenterazine analogs were screened to optimize the substrate. (Dale et al., Front. Bioeng. Biotechnol. 2019, 7, 56.)

Examples of additional luciferase donor/substrates include teLuc and diphenylterazine. TeLuc and diphenylterazine are derivatives of Nluc and coelenterazine. (Dale et al., Front. Bioeng. Biotechnol. 2019, 7, 56.)

Examples of additional fluorophores and a fluorophore system includes boron-dipyrromethene (BODIPY), alprenolol-tetramethylrhodamine (TAMRA), 4-nitro-7-aminobenzofurazan, Alexa Fluor™ and the HaloTag® fluorophore system. The HaloTag® system is made up of a small halotag protein (33 kDa) fused to a chosen protein and a chloroalkane linker, wherein the linker is joined to a fluorophore. (Dale et al., Front. Bioeng. Biotechnol. 2019, 7, 56.)

In certain embodiments, the BRET assembly comprising the luciferase donor conjugated to the fluorophore acceptor is selected from the following combinations: NLuc-HT/HL-Oregon green, RLuc/YFP, RLuc/florescent protein (GFP), RLuc8/GFP, firefly luciferase/DsRed, RLuc/ODot, Rluc8/ODot, and NanoLuc/Halotag-florescent ligand.

### I.C. FRET Assembly Complex

The FRET assembly complex comprise a donor fluorophore joined to an acceptor fluorophore through a fluorophore donor-acceptor linker. Following excitation of the donor fluorophore, energy is transferred from the donor to excite the fluorophore acceptor. Emission from the donor and acceptor fluorophores can be measured. Emission can be characterized by, for example, wavelength, intensity, lifetime, and polarization.

Energy transfer between the donor fluorophore and acceptor fluorophore occurs when the donor and acceptor are within approximately <10 nm of each other. (Bajar et al., Sensors (Basel) 2016 Sep. 14; 16 (9): 1488.) The closer the donor and acceptor are to each other the stronger the emission. In different embodiments, the distance is about 1 nm, about 2 nm, about 3 nm, about 4 nm, about 5 nm, about 6 nm, about 7 nm, about 8 nm, or about 10 nm.

The fluorophore donor-acceptor linker can include atoms such as carbon, nitrogen, silicone, oxygen, sulfur, fluorine, hydrogen, and phosphorous.

The linker can comprise different types of groups or polymers such as PEG, polyaminoacids, polyacrylamides, polyvinylpyrrolidon, zwitterionic polymers, polysaccharides, poly(N-(2-hydroxypropyl) methacrylamide), poly(oligo (ethylene glycol) methyl ether methacrylate), carboxylic dextran, hydrocarbon chains and substituted hydrocarbon chains.

Multiple combinations of FRET donor and acceptor fluorophores have been developed. Examples of different FRET pairs include: cyan florescent protein and yellow florescent protein; green florescent protein and red florescent protein; and far-red florescent protein and infrared florescent protein. Examples of cyan florescent protein and yellow florescent protein include Aquamarine, ECFP, mTurquoise2, mCerulean3, mTFP1, EYFP, m Venus, SEYFP, mCitrine, and YPet. Examples of green florescent protein and red florescent protein include EGFP, NowGFP, Clover, mClover3, mNeonGreen, mRuby2, mRuby3 and mCherry. Examples of far-red florescent protein and infrared florescent protein include mPlum, eqFP650 and mCardinal. (Bajar et al. florescent protein hypertext://doi.org/10.3390/s16091488, hereby incorporated by reference herein in its entirety.)

### I.D. Signal Amplifier Localized Surface Plasmon Resonance Nanostructure

The signal amplifier localized surface plasmon resonance nanostructure can move in solution allowing the analyte binding molecule to contact and bind to analyte. Preferably, the signal amplifier is soluble in the sample being tested.

The signal amplifier can comprise different plasmonic material and can be in different shapes and sizes. Examples of plasmonic material include metals, such as europium, gold, silver, copper, palladium and alumni; and non-metals, for example, graphene, silica, and carbon nanotube.

In certain embodiments, the plasmonic material comprises metals such as rhenium, ruthenium, rhodium, palladium, silver, osmium, iridium, platinum, gold and alumni; and combinations of materials. (See, for example, Park et al. Biosensors (Basel), 2022, 17; 12 (3):180.doi:10.3390/bios 12030180; and Osborne and Pikramenou (2015) Faraday Diss., 185:291.) Examples of combinations include nickel, gold-silver alloy nanoparticles (Au—Ag), gold-palladium alloy nanoparticles (Au—Pd), gold-copper alloy nanoparticles (Au—Cu), silver-copper alloy nanoparticles (Ag—Cu), gold-silver-copper alloy nanoparticles (Au—Ag—Cu), gold-silver-palladium alloy nanoparticles (Au—Ag—Pd), metal oxide nanoparticles (e.g., titanium dioxide and zinc oxide), and metal-semiconductor nanoparticles (e.g., gold-silicon and silver-silicon).

The localized surface plasmon resonance nanostructure can enhance the BRET and FRET signals. The closer the localized surface plasmon resonance nanostructure to the BRET and FRET assembly complex the greater the enhancement. Preferably, the localized surface plasmon resonance nanostructure is within about 5 to about 20 nm of the BRET or FRET assembly complex. In certain embodiments the localized surface plasmon resonance nanostructure is 12, 13, 14, 15, 16 nm from the BRET or FRET assembly complex.

### II. Localized Surface Plasmon Resonance Sensor

The localized surface plasmon resonance sensor comprises an analyte capture molecule coupled to a localized surface plasmon resonance nanostructure. Binding of the analyte to sensor produces a detectable change in the plasmon resonance. In some embodiments, the sensor localized surface plasmon resonance nanostructure may also interact with the BRET assembly complex, the FRET assembly complex and/or the amplifier localized surface plasmon resonance nanostructure.

### II.B. Analyte Capture Molecule

The analyte capture molecule can be used in combination with an analyte binding molecule, where both bind to the same analyte. Examples of different types of analytes are noted in Section I. Like analyte binding molecules, analyte capture binding molecules can bind to a variety of different analytes. While the particular type of analyte capture molecule (e.g., polynucleotide or protein) may be the same or different than an analyte binding molecule binding to the same analyte, the analyte capture and analyte binding molecule combination should be selected to bind to different parts of an analyte so as not to interfering with each other.

Analyte capture molecules are able to specifically bind to an analyte of interest. Specific binding refers to the ability to bind to the analyte based upon the particular structure of the analyte, and distinguish (e.g., binds to a significantly greater extent) the targeted analyte from other analytes naturally present in a sample (e.g., biological sample). Absolute specificity while very helpful for some applications, may not be required. For example, the analyte capture molecule can be used in conjunction with a binding molecule, both of which are specific for an analyte and assist in detecting the presence of the analyte. In different embodiments the capture molecules has a specificity to a target analyte at least 10×, at 100× or at least 1000× greater than other analytes present in the sample being tested.

In certain embodiments, the analyte capture molecule is a single-stranded capture oligonucleotide. A single-stranded capture oligonucleotide comprises purine or pyrimidine nucleobases or derivatives thereof, able to hydrogen bond via Watson-Crick hydrogen bonds with nucleobases present in DNA or RNA.

Various modifications can be made to the different polynucleotides components to provide for capture oligonucleotide molecules able to hydrogen bond to RNA and DNA having complementary nucleotide sequences. Examples of purine modifications include 2,6-diaminopurine, 3-deaza-adenine, 7-deasa-guanine, and 8-azido adenine. Examples of pyrimidine modifications include 2-thio-thymidine, 5-carboxamine-uracil, 5-methyl-cytosine, 3-ethynyl-uracil. Examples of phosphodiester modifications include methylphosphonate, phosphorothioate, and guanidinopropyl phosphoramidate. Examples of phosphate replacement includes triazole and guanidinium. Examples of sugar modifications include 2′-modifications such as 2′-F, 2′-methoxy, 2′-amino, and 2′-azido; locked sugar; 3′ end modifications; and 5′ end modification. (Ochoa and Milam, Molecules 2000, 25, 4689, hereby incorporated reference herein in its entirety.)

Another example of a modification is peptide nucleic acid, where the sugar-phosphate backbone is replaced with a neutral pseudopeptide backbone. The peptide nucleic acid retains nucleobase complementarity, and the ability to hybridize to naturally occurring DNA and RNA. Peptide nucleic acid can be produced, for example, by replacing the phosphodiester backbone with N-(2 aminoethyl) glycine, wherein the nucleobases are connected to the backbone via a methylene carbonyl linker. Additional peptide nucleic acid structures and design considerations are provided in Brodyagin et al., J. Org. Chem. 2021, 17, 1641-1688; and Moccia et al., Artif. DNA: PNA& XNA. 2014 5 (3): e1107176; each of which are hereby incorporated by reference herein in their entirety.

Oligonucleotide capture molecules can vary in size and degree of complementarity to target DNA or RNA. The degree of complementarity providing for specificity will vary depending on oligonucleotide structure, for example purine versus pyrimidine nucleobases, and modifications to the nucleobase, sugar and/or phosphodiester group; and reaction conditions. In different embodiments, concerning the capture oligonucleotide molecule size, the oligonucleotide comprises at least 10, at least 12, at least 15, at least 20, at least 30, at least 40, or at least 50, nucleobases. In different embodiments concerning the degree of complementarity to a target nucleic acid analyte, the oligonucleotide capture molecule comprises a region of 10 or more, 11 or more, 12 or more, 13 or more, 15 or more, 16 or more, 17 or more, 18 or more, 19 or more, 20 or more, 21 or more, 22 or more, 23 or more, 24 or more, or 25 or more nucleobases with at least 90%, at least 95% or 100% complementarity to a target nucleic acid analyte.

In certain embodiments the analyte capture molecule is an antibody or comprises a binding fragment of an antibody. Antibody binding fragments contain three complementary determining regions in a variable region framework allowing for antigen binding. Examples of binding fragments include FAb fragments, single chain variable region fragments (scFV), single domain fragments (dAbs), Fv fragments, camelid heavy-chain variable domains (VHHs), mini-body and diabody.

In certain embodiments the analyte capture molecule is an aptamer.

In certain embodiments the analyte capture molecule is a protein. Different types of protein can be used to bind to analytes, such as an enzyme for binding to a substrate, a substrate for binding to an enzyme, a receptor for binding to ligand, and a ligand for binding to a receptor.

In certain embodiments the analyte capture molecule is a ligand.

The analyte capture molecule can be anchored directly to the localized surface plasmon resonance nanostructure or can be anchored through a linker. Depending on the localized surface plasmon resonance nanostructure, anchoring can be achieved through electrostatic interaction or covalent bonds.

The length and composition of the analyte capture molecule linker can vary. The individual atoms of the linker can include atoms such as carbon, nitrogen, silicone, oxygen, sulfur, hydrogen, fluorine, and phosphorous.

The linker can comprise different types of groups or polymers such as polyethylene glycol (PEG), polyaminoacids, polyacrylamides, polyvinylpyrrolidon, zwitterionic polymers, polysaccharides, poly(N-(2-hydroxypropyl) methacrylamide), poly(oligo (ethylene glycol) methyl ether methacrylate), carboxylic dextran, hydrocarbon chains and substituted hydrocarbon chains.

The linker provides a stable structure and can be made up of different groups joined together by different types of molecular interaction, and can be produced using different techniques. Examples of different techniques for joining different groups include EDC/NHS coupling, click chemistry, streptavidin-biotin and complementary oligonucleotides.

In certain embodiments, the linker comprises PEG. Different types of PEG can be employed such as linear and branched of varying size. In certain embodiments the PEG is less than about 5K. The Examples provided below illustrate a mixture of PEG-SH (1K), having COOH and NH₂ functional groups.

In certain embodiments the PEG links directly to the capture molecule where it acts as a linker (anchors to the nanoparticle) and spacer.

In certain embodiments the PEG is a spacer and is attached to the nanoparticle through another moiety. For example, the linker has a C3-SH moiety attached directly to the nanoparticles and is immobilized onto nanoparticles via Au—S bond.

### II.C. Sensor Localized Surface Plasmon Resonance Nanostructure

Surface plasmon resonance is generated by an electromatic surface wave, due to a collective oscillation of free electrons propagating parallel to an interface region. The binding of analytes to the material generates surface plasmon resonance leading to a change in refractive index, which can be identified as a wavelength shift or surface plasmon resonance intensity. Different types of plasmonic phenomena such as surface plasmon resonance, localized surface plasmon resonance, and surface-enhanced Raman scattering can be utilized for analyte detection. References describing surface plasmon resonance, detection techniques, materials, and configurations include Park et al. Biosensors (Basel), 2022, 17; 12(3):180, doi:10.3390/bios12030180; Camarca et al., Sensor 2021, 21, 906, hypertext//doi.org//10.3390/s2103906; Kim et al., Sensors 2021, 21, 3191, hypertext//doi.org//10.3390/s21093191; and Liu and Zhang, Micromachines 2021, 12, 826, hypertext//doi.org/10.3990/mil2070826; each of which are hereby incorporated by its entirety herein.

Plasmons can be classified as bulk, surface and localized surface (nanoparticles). Localized surface plasmon, is confined and excited on sub-wavelength size nanoparticles with a specific frequency known as localized surface plasmon resonance. The localized surface plasmon plasmonic nanostructure is affected by the morphology, size, composition, and distance between adjacent nanostructures. Examples of different shapes include sphere, prisms, spikes, stars, and rods.

Localized surface plasmon resonance nanoparticles can be based on different platforms and made of different materials. The nanoparticle, for example, can comprise metals such as rhenium, ruthenium, rhodium, palladium, silver, osmium, iridium, platinum, gold and alumni; and combinations of materials. (See, for example, Park et al. Biosensors (Basel), 2022, 17; 12(3):180.doi:10.3390/bios12030180; and Osborne and Pikramenou (2015) Faraday Diss., 185:291.) Examples of combinations include nickel, gold-silver alloy nanoparticles (Au—Ag), gold-palladium alloy nanoparticles (Au—Pd), gold-copper alloy nanoparticles (Au—Cu), silver-copper alloy nanoparticles (Ag—Cu), gold-silver-copper alloy nanoparticles (Au—Ag—Cu), gold-silver-palladium alloy nanoparticles (Au—Ag—Pd), metal oxide nanoparticles (e.g., titanium dioxide and zinc oxide), and metal-semiconductor nanoparticles (e.g., gold-silicon and silver-silicon).

In certain embodiments, the localized surface plasmon resonance nanoparticles are provided on a substrate such as fiber, a fiber array, or a disk; and/or patterned plasmonic assay-based plasmonic sensor such as a nanoparticle structure array, and nanohole or cavity array substrate. The plasmonic sensing system can be integrated on a microfluidic platform.

The substrate can be anchored directly to the localized surface plasmon resonance nanostructure or can be anchored through a linker. Depending on the localized surface plasmon resonance nanostructure, anchoring can be achieved through electrostatic interaction or covalent bonds. In certain embodiments, the linker joined to the nanoparticle comprises a group provided in Table 2. Table 2 compounds referencing “n”, provide for n=1 to n=10,000.

TABLE 2

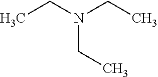

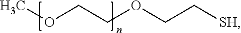

PEG(n)

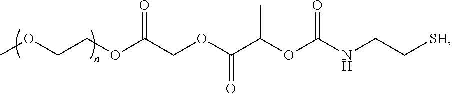

mPEG(n)-PLGA-SH wherein

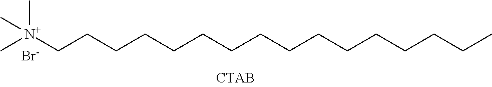 [SMILES: CCCCCCCCCCCCCCCC[N+](C)(C)C ; [Br-]]

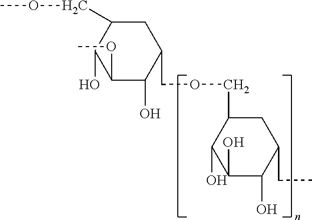

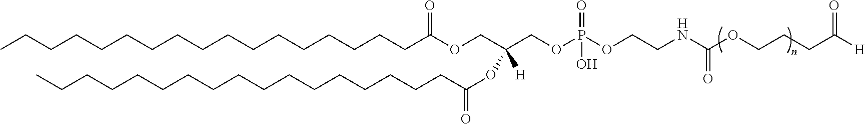

DSPE-PEG(n)-Ald

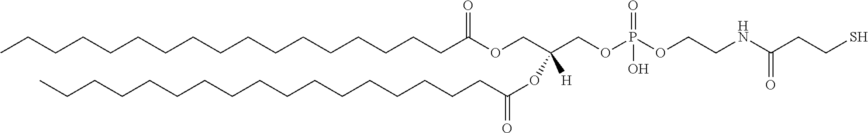 [SMILES: CCCCCCCCCCCCCCCCCC(=O)OC[C@H](COP(=O)(O)OCCNC(=O)CCS)OC(=O)CCCCCCCCCCCCCCCCC]

DSPE

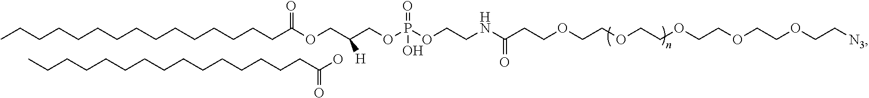

DSPE-PEG(n)-azide

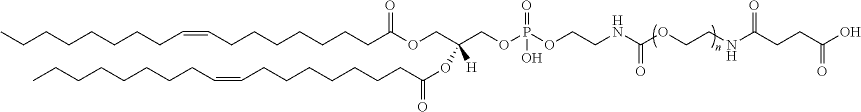

DOPE-PEG(n)-COOH

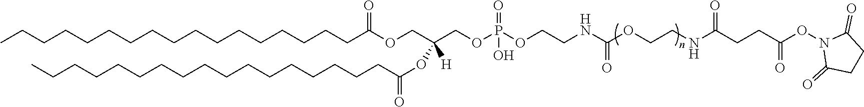

DSPE-PEG(n)-NHS

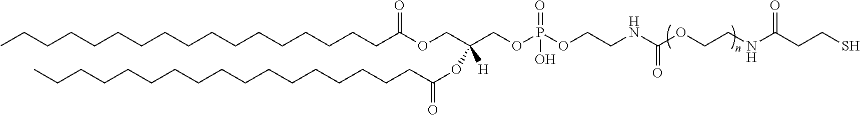

DSPE-PEG(n)-SH

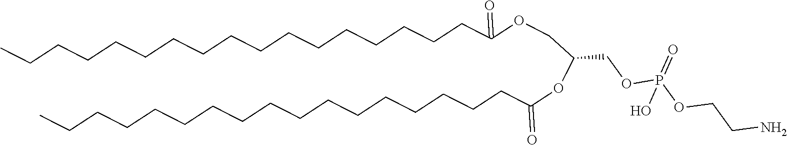 [SMILES: CCCCCCCCCCCCCCCCCC(=O)OC[C@H](COP(=O)(O)OCCN)OC(=O)CCCCCCCCCCCCCCCCC]

1,2-Distearoyl-sn-glycero-3-phosphoethanolamine

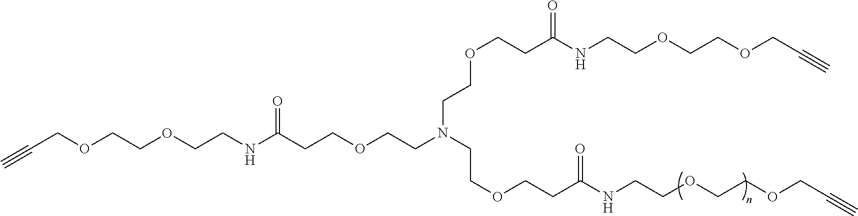

Tri(propargyl-PEG(n)-NHCO-ethyloxyethyl)amine

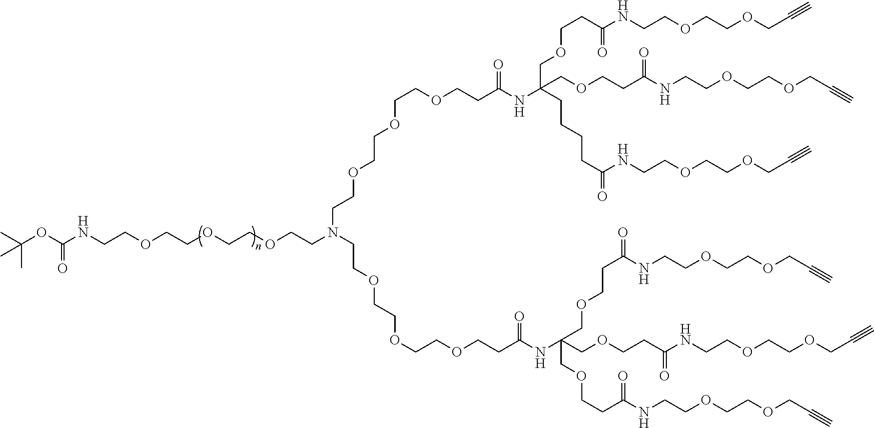

N-(Boc-PEG3)-N-bis-(PEG(n)-Amino-Tri-(Propargyl-PEG(n)-ethoxymethyl)-methane)

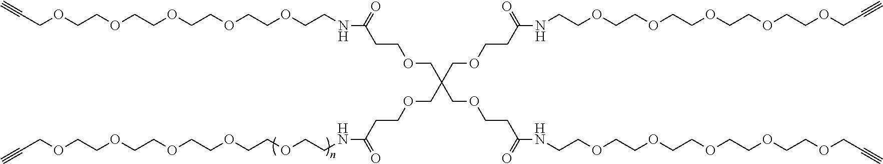

Tetra(3-methoxy-N-(PEG(n)-prop-2-ynyl)propanamide)Methane

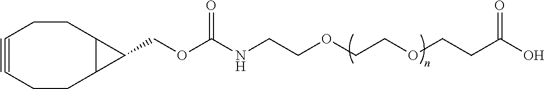

endo-BCN-PEG(n)-acid

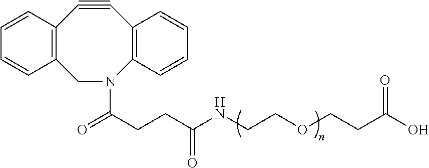

DBCO-PEG(n)-acid

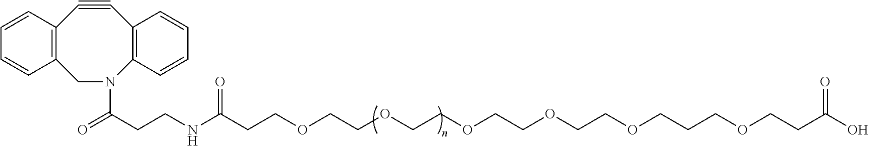

DBCO-NHCO-PEG(n)-acid

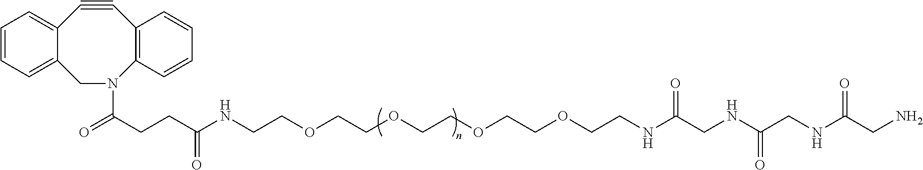

Gly-Gly-Gly-PEG(n)-DBCO

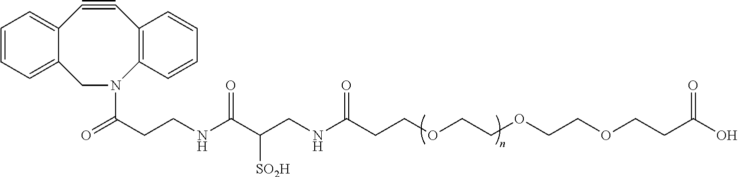

Sulfo DBCO-PEG(n)-acid

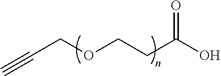

Propargyl-PEG(n)-acid

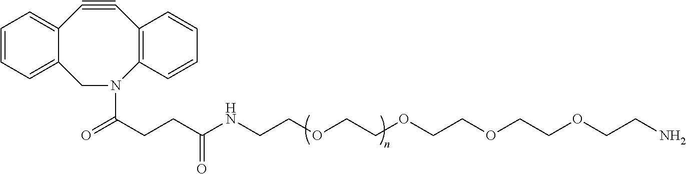

DBCO-PEG(n)-amine TFA salt

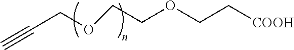

Propargyl-PEG(n)-CH2CO2H

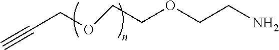

Propargyl-PEG(n)-amine

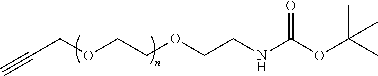

t-Boc-N-Amido-PEG(n)-propargyl

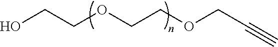

Propargyl-PEG(n)-alcohol

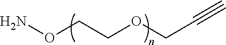

Aminooxy-PEG1-propargyl HCl salt

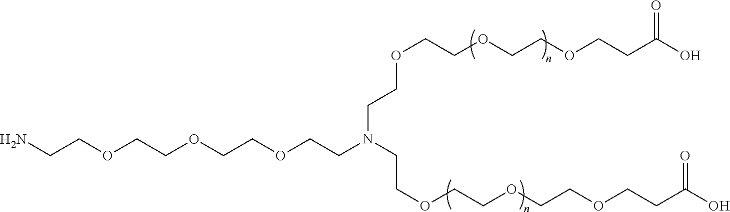

N-(Amino-PEGn)-N-bis(PEG3-acid)

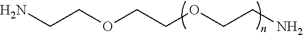

Amino-PEG(n)-amine

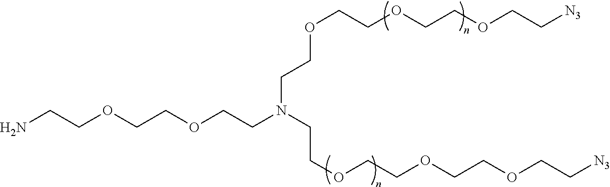

N-(Amino-PEG(n))-N-bis(PEG3-azide)

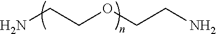

m-PEG2-amine

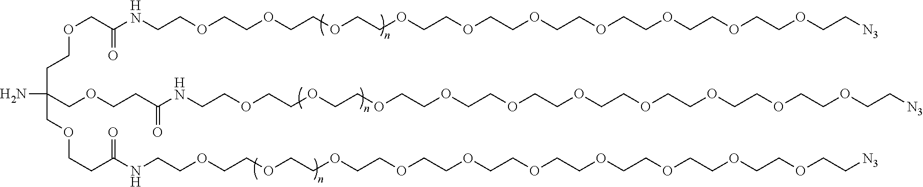

Amino-Tri-(Azide-PEG(n)-ethoxymethyl)-methane

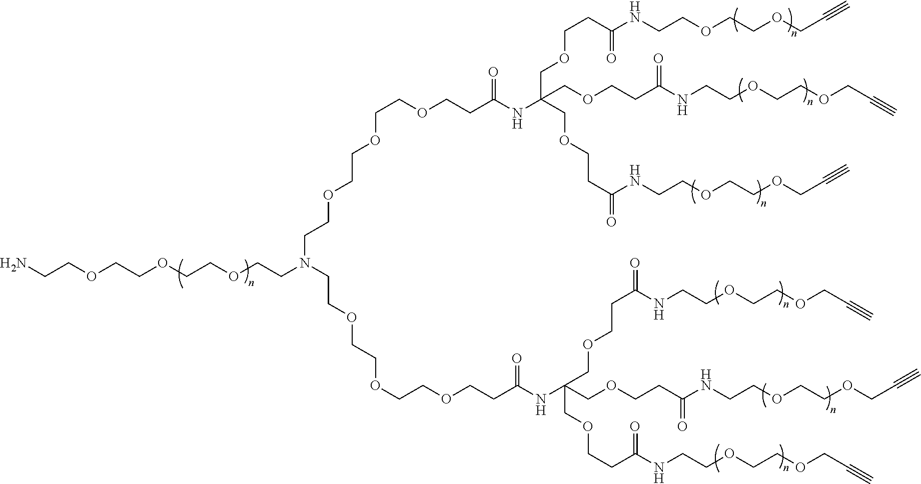

N-(Amine-PEG(n))-N-bis-(PEG(n)-Amino-Tri-(Propargyl-PEG(n)-ethoxymethyl)-
methane)

In certain embodiments, binding to the nanoparticle is through a thiol, phosphene, amine, a polymer, or silica. (See, e.g., Mahota et al., (2019) 3 Biotech 9:57, illustrating functionalization using gold nanoparticles.)

In certain embodiments the nanoparticle is functionalized with a silane modifier. Silane modifiers are described by, for example, Ahangaran and Navarchian (2020) Advances in Colloid and Interface Science 286:102209.

Localized surface plasmon resonance can have a variety of different uses such as being using to confirm functionalization of the nanostructure, amplifying signal produced from the BRET or FRET assembly complex, and/or measuring analyte binding. Amplification of the BRET or FRET signals is affected by the distance between the surface localized plasmon resonance nanostructure and the BRET or FRET assembly complex. Preferably, the distance should be no greater than about 15 nm.

In certain embodiments, (a) the sensor localized surface plasmon resonance signal or surface-enhanced Raman scattering and (b) the amplifier localized surface plasmon resonance or Ramen scattering, may enhance each other's signals. In further embodiments enhancement is achieved when the sensor providing a localized surface plasmon resonance signal and the amplifier providing a localized surface plasmon resonance are within about 30 nm.

### III. Analyte Detection Assays

The amplifiers and sensors described herein can be used to detect a variety of different analytes from different samples. Assay formats can vary depending upon the sample and the use of a BRET or FRET assembly complex. Various type of samples can be assayed including biological, food, agricultural, and environmental.

A basic assay involving the use of a BRET assembly complex comprises: (a) capturing an analyte on to sensor; (b) providing the amplifier; (c) adding a luciferase substrate; and (d) measuring fluorescence, localized surface plasmon resonance, bioluminescence, or surface-enhanced Raman scattering. More than one readout can be measured. Unless otherwise indicated, reference to steps (a) and (b) do not provide for a particular order. In certain embodiments, step (a) is followed by step (b). In certain embodiments, step (b) is followed by step (a).

In different embodiments the analyte is quantified based on two or more different readouts selected from fluorescence, bioluminescence, localized surface plasmon resonance and surface-enhanced Raman scattering; or measuring at least fluorescence and/or bioluminescence. Dual techniques can increase overall accuracy, to confirm a positive result, and are particularly useful where low amounts of analyte are present.

A basic assay involving the use of a FRET assembly complex comprises: (a) capturing an analyte on to sensor; (b) providing the amplifier; (c) exciting the fluorophore; and (d) measuring fluorescence or surface-enhanced Raman scattering. More than one readout can be measured. Unless otherwise indicated, reference to steps (a) and (b) do not provide for a particular order. In certain embodiments, step (a) is followed by step (b). In certain embodiments, step (b) is followed by step (a).

In certain embodiments, unbound amplifier is removed prior to the measuring step.

In certain embodiments, analyte from a particular sample are either purified or not purified prior to the assay; and/or analyte from a particular sample are either amplified or not amplified prior to the assay.

### IV. Bladder Cancer Analyte Detection and Treatment

Bladder cancer is a deadly and highly recurrent disease that requires extensive routine follow-up testing to ensure progression is caught early. Because of its chronic nature, bladder cancer requires extensive routine testing to monitor for disease recurrence and progression, especially during the first two years post-diagnosis. (Chamie et al., Cancer 2011, 117 (23), 5392-401.) Early bladder detection also significantly improves patient prognosis. (Shokeir, B J U Int 2004, 93 (2), 216-20.)

### IV.A. Bladder Cancer Analyte Detection

The following analytes were identified as providing a combination of analytes useful in bladder cancer diagnose and treatment: miR-200C, miR-205, miR-16-1, miR-143, UCA1 nucleic acid, insulin-like growth factor 2 (IGF2) or IGF2 nucleic acid; Annexin-10 (ANXA10) or ANXA10 nucleic acid, nuclear mitotic apparatus protein (NMP-22) or NMP-22 nucleic acid, human complement factor H-related protein (HCFHrp) or HCFHrp nucleic acid, Uroplakin (UPK1B) or UPK1B nucleic acid, ABL1 tyrosine kinase (ABL1) or ABL1 nucleic acid, CRH (corticotropin) or CRH nucleic acid, and P53 or P53 nucleic acid. Additional analytes can be tested. An example of an additional analyte is keratin, type I cytoskeletal 17 (KRT17) or KRT17 nucleic acid. Reference to nucleic acid for a particular analyte provides for related nucleic acid and includes different forms such as RNA and DNA, and different types of RNA and DNA.

Expression of miR-143 is downregulated in human bladder cancer tissues and cells and is considered a tumor suppressor microRNA. Its functional target is the insulin-like growth factor-1 receptor (IGF-1R). Overexpression of miR-143 inhibits cell proliferation and promote chemosensitivity of bladder cancer 5637 cells to gemcitabine. (Wang et al., Oncol. Lett. 2017, 13 (1) 435-440.)

MicroRNA 205 is an oncogenic microRNA and has a role in the inhibition of proliferation, migration, and invasion of bladder cancer cells. (Sun et al, Cell Death & Disease 2015, 6 (10), e1907-e1907.)

MicroRNA 200c acts as tumor suppressor microRNA and negatively regulates angiogenesis in bladder cancer and further negatively regulates the expression of angiogenesis-related proteins, such as HIF-1α and VEGF3. (Wu et al., Translational Cancer Research 2019, 8 (8), 2713-2724.)

MicroRNA 16-1 is downregulated in bladder cancer compared to the adjacent normal tissues and acts as a tumor suppressor microRNA. (Jiang et al., Asian Pac J Cancer Prev. 2013; 14(7):4127-30.)

Overexpression of lncRNA-UCA1 induces epithelial-mesenchymal transition (EMT) and increases bladder cancer cells' migratory and invasive abilities. (Xue et al., Cancer Sci 2016, 107 (1), 18-27.)

Accession numbers for human IGF2, ANXA10, NMP-22, HCFHrp, UPK1B, ABL1, CRH, P53, and KRT17, are provided in Table 3. CRH is part of a family that includes the corticotropin-releasing hormone (CRH) homologous urocortin proteins (UCN, UCN2, UCN3), their receptors CRHR1 and CRHR2 as well as the corticotropin-releasing hormone-binding protein CRHRB. CRH plays a role in the development of solid human cancers. IGF2 is a tumor promoter that drives cancer proliferation through its client IGF2 mRNA and HMGA16 mRNA. (Dai et al., eLife 2017, 6, e27155.) ABL1-MS1 is passed on to offspring according to Mendelian inheritance through meiosis. The ABL1-MS1 region can affect ABL1 expression in bladder cancer. (Kim et al. BMC Medical Genomics 2021, 14, 121.) UPK1B is upregulated in bladder cancer and is significantly correlated with tumor stage, lymph node metastasis, distant metastasis, and poor prognosis of bladder cancer. (Wang et al., Eur Rev Med Pharmacol Sci. 2018 September; 22 (17): 5471-5480.) ANXA plays a role in the regulation of cellular growth and signal transduction pathway, and down-regulation of ANXA10 in a bladder cancer cell line induced increased proliferation and migration. (Munksgaard et al., British Journal of Cancer 2011, 105.) Nuclear matrix proteins relate to the cell's genetic expression and response to various xenobiotics stimuli. (Szymańska et al., Biomed Res Int 2017, 2017, 9643139-9643139.) (See also, Xylinas et al, Urol Oncol 2014, 32 (3), 222-9; and Oge et al., Urol Nephrol. 2001; 32(3):367-70.) Keratin, type I cytoskeletal 17 (KRT17) is over expressed in some cancers and increased levels has been indicated to be a biomarker for bladder cancer. (U.S. Pat. No. 11,360,092.)

Table 3 provides different accession numbers for the different proteins, along with examples of protein and nucleic acid accession numbers. Different variants such as different protein isoforms and different encoding nucleic acid exist. The information associated with each of the provided accession numbers are hereby incorporated by reference herein in its entirety.

TABLE 3

| | Accession Number | DNA Accession Numbers | |
|---|---|---|---|
| IGF2 | P01344: | Genomic: X03562, | hypertext://www.uniprot.org/ |
| | MGIPMGKSMLVLLTFLAFASCCIA | X03425, X03426, X03427 | uniprotkb/P01344/entry |
| | AYRPSETLCGGELVDTLQFVCGD | mRNA: X00910, J03242 | |
| | RGFYFSRPASRVSRRSRGIVEECC | | |
| | FRSCDLALLETYCATPAKSERDVS | | |
| | TPPTVLPDNFPRYPVGKFFQYDT | | |
| | WKQSTQRLRRGLPALLRARRGH | | |
| | VLAKELEAFREAKRHRPLIALPTQ | | |
| | DPAHGGAPPEMASNRK | | |
| | SEQ ID NO: 1 | | |
| ANXA10 | Q9UJ72: | mRNA: AJ238979, | hypertext://www.uniprot.org/ |
| | MFCGDYVQGTIFPAPNFNPIMDA | AF196478, BC007320 | uniprotkb/Q9UJ72/entry |
| | QMLGGALQGFDCDKDMLINILTQ | | |
| | RCNAQRMMIAEAYQSMYGRDLI | | |
| | GDMREQLSDHFKDVMAGLMYPP | | |
| | PLYDAHELWHAMKGVGTDENCL | | |
| | IEILASRINGEIFQMREAYCLQYS | | |
| | NNLQEDIYSETSGHFRDTLMNLV | | |
| | QGTREEGYTDPAMAAQDAMVL | | |
| | WEACQQKTGEHKTMLQMILCNK | | |
| | SYQQLRLVFQEFQNISGQDMVDA | | |
| | INECYDGYFQELLVAIVLCVRDKP | | |
| | AYFAYRLYSAIHDFGFHNKTVIRI | | |
| | LIARSEIDLLTIRKRYKERYGKSLF | | |
| | HDIRNFASGHYKKALLAICAGDA | | |
| | EDY | | |
| | SEQ ID NO: 2 | | |
| NMP-22 | Q14980 | Genomic: CH471076 | hypertext://www.uniprot.org/ |
| | MTLHATRGAALLSWVNSLHVAD | mRNA: Z11583, Z11584, | uniprotkb/Q14980/entry |
| | PVEAVLQLQDCSIFIKIIDRIHGTE | BC004165 | |
| | EGQQILKQPVSERLDFVCSFLQKN | | |
| | RKHPSSPECLVSAQKVLEGSELEL | | |
| | AKMTMLLLYHSTMSSKSPRDWE | | |
| | QFEYKIQAELAVILKFVLDHEDGL | | |
| | NLNEDLENFLQKAPVPSTCSSTFP | | |
| | EELSPPSHQAKREIRFLELQKVAS | | |
| | SSSGNNFLSGSPASPMGDILQTPQ | | |
| | FQMRRLKKQLADERSNRDELELE | | |
| | LAENRKLLTEKDAQIAMMQQRID | | |
| | RLALLNEKQAASPLEPKELEELRD | | |
| | KNESLTMRLHETLKQCQDLKTEK | | |
| | SQMDRKINQLSEENGDLSFKLRE | | |
| | FASHLQQLQDALNELTEEHSKAT | | |
| | QEWLEKQAQLEKELSAALQDKK | | |
| | CLEEKNEILQGKLSQLEEHLSQLQ | | |
| | DNPPQEKGEVLGDVLQLETLKQ | | |
| | EAATLAANNTQLQARVEMLETE | | |
| | RGQQEAKLLAERGHFEEEKQQLS | | |
| | SLITDLQSSISNLSQAKEELEQASQ | | |
| | AHGARLTAQVASLTSELTTLNATI | | |
| | QQQDQELAGLKQQAKEKQAQLA | | |
| | QTLQQQEQASQGLRHQVEQLSSS | | |
| | LKQKEQQLKEVAEKQEATRQDH | | |
| | AQQLATAAEEREASLRERDAALK | | |
| | QLEALEKEKAAKLEILQQQLQVA | | |
| | NEARDSAQTSVTQAQREKAELSR | | |
| | KVEELQACVETARQEQHEAQAQ | | |
| | VAELELQLRSEQQKATEKERVAQ | | |
| | EKDQLQEQLQALKESLKVTKGSL | | |
| | EEEKRRAADALEEQQRCISELKA | | |
| | ETRSLVEQHKRERKELEEERAGR | | |
| | KGLEARLQQLGEAHQAETEVLRR | | |
| | ELAEAMAAQHTAESECEQLVKE | | |
| | VAAWRERYEDSQQEEAQYGAMF | | |
| | QEQLMTLKEECEKARQELQEAKE | | |
| | KVAGIESHSELQISRQQNELAELH | | |
| | ANLARALQQVQEKEVRAQKLAD | | |
| | DLSTLQEKMAATSKEVARLETLV | | |
| | RKAGEQQETASRELVKEPARAGD | | |
| | RQPEWLEEQQGRQFCSTQAALQA | | |
| | MEREAEQMGNELERLRAALMES | | |
| | QGQQQEERGQQEREVARLTQER | | |
| | GRAQADLALEKAARAELEMRLQ | | |
| | NALNEQRVEFATLQEALAHALTE | | |
| | KEGKDQELAKLRGLEAAQIKEL | | |
| | EELRQTVKQLKEQLAKKEKEHAS | | |
| | GSGAQSEAAGRTEPTGPKLEALR | | |
| | AEVSKLEQQCQKQQEQADSLERS | | |
| | LEAERASRAERDSALETLQGQLE | | |
| | EKAQELGHSQSALASAQRELAAF | | |
| | RTKVQDHSKAEDEWKAQVARGR | | |
| | QEAERKNSLISSLEEEVSILNRQVL | | |
| | EKEGESKELKRLVMAESEKSQKL | | |
| | EERLRLLQAETASNSARAAERSS | | |
| | ALREEVQSLREEAEKQRVASENL | | |
| | RQELTSQAERAEELGQELKAWQE | | |
| | KFFQKEQALSTLQLEHTSTQALVS | | |
| | ELLPAKHLCQQLQAEQAAAEKR | | |
| | HREELEQSKQAAGGLRAELLRAQ | | |
| | RELGELIPLRQKVAEQERTAQQL | | |
| | RAEKASYAEQLSMLKKAHGLLA | | |
| | EENRGLGERANLGRQFLEVELDQ | | |
| | AREKYVQELAAVRADAETRLAE | | |
| | VQREAQSTARELEVMTAKYEGA | | |
| | KVKVLEERQRFQEERQKLTAQVE | | |
| | QLEVFQREQTKQVEELSKKLAD | | |
| | SDQASKVQQQKLKAVQAQGGES | | |
| | QQEAQRLQAQLNELQAQLSQKE | | |
| | QAAEHYKLQMEKAKTHYDAKK | | |
| | QQNQELQEQLRSLEQLQKENKEL | | |
| | RAEAERLGHELQQAGLKTKEAEQ | | |
| | TCRHLTAQVRSLEAQVAHADQQ | | |
| | LRDLGKFQVATDALKSREPQAKP | | |
| | QLDLSIDSLDLSCEEGTPLSITSK | | |
| | LPRTQPDGTSVPGEPASPISQRLPP | | |
| | KVESLESLYFTPIPARSQAPLESSL | | |
| | DSLGDVFLDSGRKTRSARRRTTQI | | |
| | INITMTKKLDVEEPDSANSSFYST | | |
| | RSAPASQASLRATSSTQSLARL | | |
| | GSPDYGNSALLSLPGYRPTTRSSA | | |
| | RRSQAGVSSGAPPGRNSFYMGTC | | |
| | QDEPEQLDDWNRIAELQQRNRVC | | |
| | PPHLKTCYPLESRPSLSLGTITDEE | | |
| | MKTGDPQETLRRASMQPIQIAEG | | |
| | TGITTRQQRKRVSLEPHQGPGTPE | | |
| | SKKATSCFPRPMTPRDRHEGRKQ | | |
| | STTEAQKKAAPASTKQADRRQS | | |
| | MAFSILNTPKKLGNSLLRRGASK | | |
| | KALSKASPNTRSGTRRSPRIATTT | | |
| | ASAATAAAIGATPRAKGKAKH | | |
| | SEQ ID NO: 3 | | |
| HCFHrp | P36980 | Genomic: X86564, | hypertext://www.uniprot.org/ |
| | MWLLVSVILISRISSVGGEAMFCD | X86565, X86566, | uniprotkb/P36980/entry |
| | FPKINHGILYDEEKYKPFSQVPTG | X86567; | |
| | EVFYYSCEYNFVSPSKSFWTRITC | mRNA: X64877, | |
| | AEEGWSPTPKCLRLCFFPFVENG | BC022283 | |
| | HSESSGQTHLEGDTVQIICNTGYR | | |
| | LQNNENNISCVERGWSTPPKCRS | | |
| | TISAEKCGPPPPIDNGDITSFLLSV | | |
| | YAPGSSVEYQCQNLYQLEGNNQI | | |
| | TCRNGQWSEPPKCLDPCVISQEIM | | |
| | EKYNIKLKWTNQQKLYSRTGDIV | | |
| | EFVCKSGYHPTKSHSFRAMCQNG | | |
| | KLVYPSCEEK | | |
| | SEQ ID NO: 4 | | |
| UPK1B | O75841 | Genomic: AF067147 | hypertext://www.uniprot.org/ |
| | MAKDNSTVRCFQGLLIFGNVIIGC | mRNA: AB015234, | uniprotkb/O75841/entry |
| | CGIALTAECIFFVSDQHSLYPLLE | AF042331, AB002155, | |
| | ATDNDDIYGAAWIGIFVGICLFCL | BC063568, AF082888 | |
| | SVLGIVGIMKSSRKILLAYFILMFI | | |
| | VYAFEVASCITAATQQDFFTPNL | | |
| | FLKQMLERYQNNSPPNNDDQWK | | |
| | NNGVTKTWDRLMLQDNCCGVN | | |
| | GPSDWQKYTSAFRTENNDADYP | | |
| | WPRQCCVMNNLKEPLNLEACKL | | |
| | GVPGFYHNQGCYELISGPMNRHA | | |
| | WGVAWFGFAILCWTFWVLLGTM | | |
| | FYWSRIEY | | |
| | SEQ ID NO: 5 | | |
| ABL1 | P00519 | Genomic: U07653, | hypertext://www.uniprot.org/ |
| | MLEICLKLVGCKSKKGLSSSSSCY | U07651, DQ145721, | uniprotkb/P00519/entry |
| | LEEALQRPVASDFEPQGLSEAAR | CH471090, S69223 | |
| | WNSKENLLAGPSENDPNLFVALY | mRNA: M14752, | |
| | DFVASGDNTLSITKGEKLRVLGY | X16416, BC117451 | |
| | NHNGEWCEAQTKNGQGWVPSNY | | |
| | ITPVNSLEKHSWYHGPVSRNAAE | | |
| | YLLSSGINGSFLVRESESSPGQRSI | | |
| | SLRYEGRVYHYRINTASDGKLYV | | |
| | SSESRFNTLAELVHHHSTVADGLI | | |
| | TTLHYPAPKRNKPTVYGVSPNYD | | |
| | KWEMERTDITMKHKLGGGQYGE | | |
| | VYEGVWKKYSLTVAVKTLKEDT | | |
| | MEVEEFLKEAAVMKEIKHPNLVQ | | |
| | LLGVCTREPPFYIITEFMTYGNLL | | |
| | DYLRECNRQEVNAVVLLYMATQI | | |
| | SSAMEYLEKKNFIHRDLAARNCL | | |
| | VGENHLVKVADFGLSRLMTGDT | | |
| | YTAHAGAKFPIKWTAPESLAYNK | | |
| | FSIKSDVWAFGVLLWEIATYGMS | | |
| | PYPGIDLSQVYELLEKDYRMERPE | | |
| | GCPEKVYELMRACWQWNPSDRP | | |
| | SFAEIHQAFETMFQESSISDEVEKE | | |
| | LGKQGVRGAVSTLLQAPELPTKT | | |
| | RTSRRAAEHRDTTDVPEMPHSKG | | |
| | QGESDPLDHEPAVSPLLPRKERGP | | |
| | PEGGLNEDERLLPKDKKTNLFSA | | |
| | LIKKKKKTAPTPPKRSSSFREMDG | | |
| | QPERRGAGEEEGRDISNGALAFTP | | |
| | LDTADPAKSPKPSNGAGVPNGAL | | |
| | RESGGSGFRSPHLWKKSSTLTSSR | | |
| | LATGEEEGGGSSSKRFLRSCSAS | | |
| | CVPHGAKDTEWRSVTLPRDLQST | | |
| | GRQFDSSTFGGHKSEKPALPRKR | | |
| | AGENRSDQVTRGTVTPPPRLVKK | | |
| | NEEAADEVFKDIMESSPGSSPPNL | | |
| | TPKPLRRQVTVAPASGLPHKEEA | | |
| | GKGSALGTPAAAEPVTPTSKAGS | | |
| | GAPGGTSKGPAEESRVRRHKHSS | | |
| | ESPGRDKGKLSRLKPAPPPPPAAS | | |
| | AGKAGGKPSQSPSQEAAGEAVLG | | |
| | AKTKATSLVDAVNSDAAKPSQPG | | |
| | EGLKKPVLPATPKPQSAKPSGTPI | | |
| | SPAPVPSTLPSASSALAGDQPSST | | |
| | AFIPLISTRVSLRKTRQPPERIASG | | |
| | AITKGVVLDSTEALCLAISRNSEQ | | |
| | MASHSAVLEAGKNLYTFCVSYV | | |
| | DSIQQMRNKFAFREAINKLENNL | | |
| | RELQICPATAGSGPAATQDFSKLL | | |
| | SSVKEISDIVQR | | |
| | SEQ ID NO: 6 | | |
| CRH | P34998 | Genomic: AF039523, | hypertext://www.uniprot.org/ |
| | MGGHPQLRLVKALLLLGLNPVSA | AF039510, AF039511, | uniprotkb/P34998/entry |
| | SLQDQHCESLSLASNISGLQCNAS | AF039512, AF039513, | |
| | VDLIGTCWPRSPAGQLVVRPCPA | AF039515, AF039516, | |
| | FFYGVRYNTTNNGYRECLANGS | AF039517, AF039518, | |
| | WAARVNYSECQEILNEEKKSKVH | AF039519, AF038520, | |
| | YHVAVIINYLGHCISLVALLVAFV | AF039521, AF039522, | |
| | LFLRLRPGCTHWGDQADGALEV | mRNA: L23332, L23333, | |
| | GAPWSGAPFQVRRSIRCLRNIIHW | X72304, AF180301, | |
| | NLISAFILRNATWFVVQLTMSPEV | FJ543100, AY45172, | |
| | HQSNVGWCRLVTAAYNYFHVTN | AB451466, AK295559, | |
| | FFWMFGEGCYLHTAIVLTYSTDR | BC096836 | |
| | LRKWMFICIGWGVPFPIIVAWAIG | | |
| | KLYYDNEKCWFGKRPGVYTDYI | | |
| | YQGPMILVLLINFIFLFNIVRILMT | | |
| | KLRASTTSETIQYRKAVKATLVLL | | |
| | PLLGITYMLFFVNPGEDEVSRVVF | | |
| | IYFNSFLESFQGFFVSVFYCFLNSE | | |
| | VRSAIRKRWHRWQDKHSIRARV | | |
| | ARAMSIPTSPTRVSFHSIKQSTAV | | |
| | SEQ ID NO: 7 | | |
| P53 | P04637 | Genomic: | |
| | MEEPQSDPSVEPPLSQETFSDLWK | M13121 M13112 M13113 | |
| | LLPENNVLSPLPSQAMDDLMLSP | M13114 M13115 M13116 | |
| | DDIEQWFTEDPGPDEAPRMPEAA | M13117 M13118 M13119 | |
| | PPVAPAPAAPTPAAPAPAPSWPLS | M13120 M22898 M22882 | |
| | SSVPSQKTYQGSYGFRLGFLHSGT | M22883 M22884 M22887 | |
| | AKSVTCTYSPALNKMFCQLAKTC | M22888 M22894 M22895 | |
| | PVQLWVDSTPPPGTRVRAMAIYK | M22896 M22897 X54156 | |
| | QSQHMTEVVRRCPHHERCSDSDG | U94788 AY838896 | |
| | LAPPQHLIRVEGNLRVEYLDDRN | AF135121 AF135120 | |
| | TFRHSVVVPYEPPEVGSDCTTIHY | AF136271 AF136270 | |
| | NYMCNSSCMGGMNRRPILTIITLE | CH471108 CH471108 | |
| | DSSGNLLGRNSFEVRVCACPGRD | AY390341 AY359814 | |
| | RRTEEENLRKKGEPHHELPPGSTK | U63714 AF209136 | |
| | RALPNNTSSSPQPKKKPLDGEYFT | AF209128 AF209129 | |
| | LQIRGRERFEMFRELNEALELKDA | AF209130 AF209131 | |
| | QAGKEPGGSRAHSSHLKSKKGQS | AF209132 AF209133 | |
| | TSRHKKLMFKTEGPDSD | AF209134 AF209135 | |
| | SEQ ID NO: 8 | AF209148 AF209149 | |
| | | AF209150 AF209151 | |
| | | AF209152 AF209153 | |
| | | AF209154 AF209155 | |
| | | AF209156 AF210309 | |
| | | AF210308 AF210310 | |
| | | AF240684 AF240685 | |
| | | AY270155 | |
| | | mRNA: | |
| | | X02469 K03199 M14694 | |
| | | M14695 X01405 X60011 | |
| | | X60012 X60013 X60014 | |
| | | X60015 X60016 X60017 | |
| | | X60018 X60019 X60020 | |
| | | AF307851 DQ186648 | |
| | | DQ186649 DQ186650 | |
| | | DQ186651 DQ186652 | |
| | | DQ191317 DQ286964 | |
| | | AB082923 AK312568 | |
| | | BC003596 AY429684 | |
| KRT17 | Q04695 | Genomic | hypertext:://www.uniprot.org/ |
| | MTTSIRQFTSSSSIKGSSGLGGGSS | Z19574 | uniprotkb/Q04695/entry |
| | RTSCRLSGGLGAGSCRLGSAGGL | mRNA | |
| | GSTLGGSSYSSCYSFGSGGGYGSS | X62571, AK095342, | |
| | FGGVDGLLAGGEKATMQNLNDR | BC000159, BC011901, | |
| | LASYLDKVRALEEANTELEVKIR | BC056421, BC072018 | |
| | DWYQRQAPGPARDYSQYYRTIEE | | |
| | LQNKILTATVDNANILLQIDNARL | | |
| | AADDFRTKFETEQALRLSVEADI | | |
| | NGLRRVLDELTLARADLEMQIEN | | |
| | LKEELAYLKKNHEEEMNALRGQ | | |
| | VGGEINVEMDAAPGVDLSRILNE | | |
| | MRDQYEKMAEKNRKDAEDWFF | | |
| | SKTEELNREVATNSELVQSGKSEI | | |
| | SELRRTMQALEIELQSQLSMKAS | | |
| | LEGNLAETENRYCVQLSQIQGLIG | | |
| | SVEEQLAQLRCEMEQQNQEYKIL | | |
| | LDVKTRLEQEIATYRRLLEGEDA | | |
| | HLTQYKKEPVTTRQVRTIVEEVQ | | |
| | DGKVISSREQVHQTTR | | |
| | SEQ ID NO: 9 | | |

The nucleic acid sequences for miR-200C, miR-205, miR-16-1, miR-143, and UCA1 are provided in Table 4, infra.

Analyte levels associated with cancer can be determined using different techniques such as comparing test sample to control samples from a bladder cancer patient, and evaluating analytes levels, for example, with information concerning analyte levels associated with bladder cancer. For example, the median NMP22 value for bladder cancer malignancies was 27.8 U/mL (95% Confidence interval: 10.5-32.1 U/mL). The median NMP22 value for the benign conditions of the bladder was 3.25 U/mL (95% Confidence interval: 2.5-3.8 U/mL). Using a reference value of 10.0 U/mL, the sensitivity of NMP22 was 100% with a specificity of 90%. (Zippe et al., Anticancer Res. 1999 July-August; 19 (4A): 2621-3.)

In comparison to healthy volunteers, patients with transitional cell carcinoma had significantly higher urinary levels of hCFHrp (117.60 vs. 2.05 U/ml; p<0.001). Using a cutoff of 17.1 U/ml, hCFHrp had a sensitivity of 72.1% and a specificity of 50.5. (Heicappell et al., Eur Urol. 1999 January; 35 (1): 81-7.)

In certain embodiments, analyte levels considered to be predictive of cancer include: NMP22 above 10 ng/ml (around 3.8 ng/ml, (14.2 pM) is present in healthy control); HCFHrp above about 10 ng/ml; mRNAs and microRNAs (oncogenic) in pM range during cancer progression (healthy controls is in the fM range); and mRNAs and microRNAs (tumor suppressor) decreased to low pM to fM range upon cancer progression (pM range for healthy controls).

In different embodiments, bladder cancer is evaluated by quantifying the presence of (a) miR-205, miR-16-1, miR-143, UCA1 nucleic acid, IGF2 or IGF2 nucleic acid, and ANXA10 or ANXA nucleic acid; (b) miR-205, miR-16-1, miR-143, UCA1 nucleic acid, IGF2 encoding mRNA, and ANXA10 encoding mRNA; (c) miR-205, miR-16-1, miR-143, UCA1 nucleic acid, IGF2 or IGF2 nucleic acid, ANXA10 or ANXA nucleic acid, NMP-22 or NMP-22 nucleic acid, HCFHrp or HCFHrp nucleic acid, miR-200C, UPKB or UPKB nucleic acid, ABL1 or ABL1 nucleic acid, and CRH or CRH nucleic acid; or (d) miR-205, miR-16-1, miR-143, UCA1 nucleic acid, IGF2 encoding mRNA, ANXA10 encoding mRNA, NMP-22 encoding mRNA, HCFHrp encoding mRNA, miR-200C, UPKB encoding mRNA, ABL1 encoding mRNA and CRH or CRH encoding mRNA. In further embodiments for (a)-(d), (i) P53 or P53 nucleic acid are quantified and/or (ii) KRT17 or KRT17 nucleic acid are quantified. In further embodiments each of the analytes in the provided combination tests positive (predictive of cancer).

In different embodiment a human patient is indicated to have bladder cancer based on analyte levels compared to that occurring in the general population involving: (a) detecting the amount of miR-205, miR-16-1, miR-143, UCA1 mRNA, IGF2 or IGF2 nucleic acid, ANXA10 or ANXA10 nucleic acid in a biological sample from the subject, where an increase in miR-205, UCA1 mRNA, IGF2 or IGF2 nucleic acid, and ANXA10 or ANXA10 nucleic acid compared to the general population indicates bladder cancer and a decrease in miR-143 and miR-16-1 compared to the general population indicates bladder cancer; (b) in addition to (a) detecting the amount of NMP-22 or NMP-22 nucleic acid, HCFHrp or HCFHrp nucleic acid, miR-200C, UPKB or UPKB nucleic acid, ABL1 or ABL1 nucleic acid, or CRH or CRH, where an increase in NMP-22 or NMP-22 nucleic acid, HCFHrp or HCFHrp nucleic acid, PKB or UPKB nucleic acid, ABL1 or ABL1 nucleic acid, or CRH or CRH nucleic acid compared to the general population indicates bladder cancer and a decrease of miR-200C compared to the general population indicates bladder cancer; (c) in addition to (a) and (b) detecting the amount of P53 or P53 encoding nucleic acid in the biological sample, where an increase P53 or P53 encoding nucleic acid compared to the general population indicates bladder cancer; and (d) in addition to (a), (b) and (c), detecting the amount of KRT17or KRT17 encoding nucleic acid in the biological sample, where an increase KRT17 or KRT17 encoding nucleic acid compared to the general population indicates bladder cancer. In further embodiments for an analyte having an increase in level associated with cancer, analyte levels of at least 2 fold, at least 5 fold, at least 10 fold or at least 25 fold compared to the average amount of the analyte in the general population are indicated to be cancer positive for that analyte; and an analyte having a decrease in level associated with cancer of 20% or less, 40%, or less, 50% or less, 75% or less, or 80% or less, compared to the average amount of the analyte in the general population are indicated to be cancer positive.

Different analytes can be detected using the same or different platforms. Reference to a particular platform refers to a substrate comprising sensors. For example, a platform can comprise a multi-well plate where different wells contain the same sensor. Alternatively, more than one type of sensor may be present. The use of localized surface plasmon nanostructures of different size, shape and/or material; along with different BRET or FRET assembly complexes can be used to facilitate using different types of sensors on the same platform.

In certain embodiments, bladder cancer analytes are detected or quantified using techniques such as those involving quantitative polymerase chain reaction, gas chromatograph, and/or flow cytometry.

In certain embodiments, bladder cancer analytes are detected or quantified using analyte detection signal amplifiers and sensors described herein.

In certain embodiments, bladder cancer analytes are quantified from a biological sample without prior analyte purification or amplification. In further embodiments, the biological sample is a urine sample.

In certain embodiments the assay is perform using a first analyte binding molecule comprising a first energy transfer molecule and a second analyte binding molecule comprising a second energy transfer molecule, wherein the first or second analyte binding molecule is attached to a fiber. The two energy transfer molecules provide for an energy transfer pair. The site where the analyte binding molecules bind, and the position of the energy transfer molecule on the binding molecules, should be selected to facilitate energy transfer. In different embodiments, the analyte binding molecules are each attached to an independently selected nanoparticle. In further embodiments, each nanoparticle is an independently selected plasmonic nanoparticle such as provided in section II.C supra.

In further embodiments, the first and second energy transfer pair is a FRET or BRET pair such as that provided in sections I.B. or I.C. supra. Preferably, the distance between FRET and BRET energy transfer pairs, when each binding molecules is bound to antigen, is within approximately <10 nm of each other. The closer the donor and acceptor are to each other the stronger the emission. In certain embodiments, the distance between energy transfer pairs are about 3 nm, about 4, nm, about 5 nm, about 6 nm, about 7 nm, about 8 nm, about 9 nm, or about 10 nm.

### IV.B. Bladder Cancer Treatment

Bladder cancer treatment can be performed and adjusted based upon measurements of the analyte combinations described in Section IV.A. Treatment includes initial treatment of patients diagnosed with bladder cancer and monitoring bladder cancer analytes; adjusting treatment, for example, increasing the therapeutic dose, continuing treatment, or discontinuing the treatment taking into account bladder cancer analytes; and treatment and monitoring following recurrence.

Bladder cancer treatment includes surgical treatment, chemotherapy, radiation therapy, immunotherapy and targeted therapy. Selection of a particular treatment regime takes into account the tumor stage, location and patient. (Riaz et al., 2021 Expert Opinion on Investigational Drugs, 30:8 837-855, hereby incorporated by reference herein.)

Approved drugs for bladder cancer treatments include atezolizumab, avelumab, erdafitinib, cisplatin, doxorubicin hydrochloride, enfortumab vedotin-ejfv, erdafitinib, mitomycin, pembrolizumab, nivolumab, sacituzumab govitecan-hziy, and valrubicin. (hypertext//www.cancer.gov/about-cancer/treatment/drugs/bladder.)

### V. Additional Aspects and Embodiments

Additional aspects, embodiments, and combinations thereof include the following:
1. An analyte detection signal amplifier comprising (a) an analyte binding molecule; (b) a localized surface plasmon resonance nanostructure and (c) either (i) a bioluminescence resonance energy transfer (BRET) assembly complex, wherein the BRET assembly complex comprises a luciferase donor conjugated to a fluorophore acceptor or (ii) a fluorescence resonance energy transfer (FRET) assembly complex, wherein the FRET assembly complex comprises a fluorescent donor conjugated to a fluorophore acceptor.
2. The analyte detection signal amplifier of 1, wherein the analyte binding molecule is either a single-stranded polynucleotide, an antibody, an aptamer, a protein, a substrate, a receptor, or a ligand.
3. The analyte detection signal amplifier of 1 or 2, wherein the surface localized plasmon resonance nanostructure is a nanorod comprises a metal.
4. The analyte detection signal amplifier of 3, wherein the nanorod comprising gold.
5. The analyte detection signal amplifier of any one of 1-4, wherein the surface localized plasmon resonance nanostructure is functionalized with polyethylene glycol (PEG), the BRET or FRET assembly complex is anchored to the localized surface plasmon resonance nanostructure by a first PEG chain, and the analyte binding molecule is anchored to the localized surface plasmon resonance nanostructure by a second PEG chain.
6. The analyte detection signal amplifier of any one of 1-5, comprising the BRET assembly complex.
7. The analyte detection signal amplifier of 6, wherein the luciferase donor conjugated to the fluorophore acceptor is selected from the following combinations: NLuc-HT/HL-Oregon green, RLuc/YFP, RLuc/florescent protein (GFP), RLuc8/GFP, firefly luciferase/DsRed, RLuc/ODot, Rluc8/ODot, or Nano Luc-HT/Halotag-florescent ligand.
8. A method of detecting or quantifying an analyte in a sample comprising the steps of:
   a) contacting a localized surface plasmon resonance sensor with the sample, wherein the sensor comprises an analyte capture molecule;
   b) providing an analyte detection signal amplifier to the sample, wherein the analyte detection signal amplifier comprises the BRET assembly complex of any one of 1-7 and binds to the analyte;
   c) adding a luciferase substrate; and
   d) detecting fluorescence, bioluminescence, localized surface plasmon resonance or surface-enhanced Raman scattering.
9. A method of detecting or quantifying an analyte in a sample comprising the steps of:
   a) contacting a localized surface plasmon resonance sensor with the sample, wherein the sensor comprises an analyte capture molecule;
   b) providing an analyte detection signal amplifier to the sample, wherein the analyte detection signal amplifier comprises the FRET assembly complex of any one of 1-5, and binds to the analyte;
   c) exciting the FRET assembly complex; and
   d) detecting fluorescence, localized surface plasmon resonance, or surface-enhanced Raman scattering.
10. The method of 8 or 9, wherein the sensor comprises a metal.
11. The method of 10, wherein the sensor comprises gold.
12. The method of any one of 8-11, wherein analyte signal amplifiers not bound to the analyte are removed after step b and prior to step c.
13. The method of any one of 8-12, wherein fluorescence and/or bioluminescence is measured.
14. The method of any one of 8-12, wherein surface-enhanced Raman scattering is measured.
15. A multi-analyte detection system comprising:
   a) a first localized surface plasmon nanostructure sensor comprising an analyte capture molecule for miR-205;
   b) a second localized surface plasmon nanostructure sensor comprising an analyte capture molecule for miR-16-1;
   c) a third localized surface plasmon nanostructure sensor comprising an analyte capture molecule for miR-143;
   d) a fourth localized surface plasmon nanostructure sensor comprising an analyte capture molecule for UCA1 nucleic acid;
   e) a fifth localized surface plasmon nanostructure sensor comprising an analyte capture molecule for IGF2 or IGF2 nucleic acid;
   f) a sixth localized surface plasmon nanostructure sensor comprising an analyte capture molecule for ANXA10 or ANXA10 nucleic acid;
      wherein each sensor type may be present on one or more platforms.
16. The multi-analyte detection system of 15, further comprising:
   g) a seventh localized surface plasmon nanostructure sensor comprising an analyte capture molecule for NMP-22 or NMP-22 nucleic acid;
   h) an eighth localized surface plasmon nanostructure sensor comprising an analyte capture molecule for HCFHrp or HCFHrp nucleic acid;
   i) a ninth localized surface plasmon nanostructure sensor comprising an analyte capture molecule for miR-200C;
   j) a tenth localized surface plasmon nanostructure sensor comprising an analyte capture molecule for UPKB or UPKB nucleic acid;
   k) an eleventh localized surface plasmon nanostructure sensor comprising an analyte capture molecule for ABL1 or ABL1 nucleic acid; and
   l) a twelfth localized surface plasmon nanostructure sensor comprising an analyte capture molecule for CRH or CRH nucleic acid; wherein each sensor type may be present on one or more platforms.
17. The multi-analyte detection system of 16, further comprising a thirteenth localized surface plasmon nanostructure sensor comprising an analyte capture molecule for P53 or P53 nucleic acid.
18. The multi-analyte detection system of 16 or 17, further comprising a fourteenth localized surface plasmon nanostructure sensor comprising an analyte capture molecule for KRT17or KRT17 nucleic acid.
19. The multi-analyte detection system of any of 15-18, wherein:
   a) the analyte capture molecule for miR-205 is a single-stranded polynucleotide complementary to miR-205;
   b) the analyte capture molecule for miR-16-1 is a single-stranded polynucleotide complementary to miR-16-1;
   c) the analyte capture molecule for miR-143 is a single-stranded polynucleotide complementary to miR-143;
   d) the analyte capture molecule for UCA1 nucleic acid is a single-stranded polynucleotide complementary to UCA1 nucleic acid;
   e) the analyte capture molecule for IGF2 or an encoding nucleic acid, is an antibody specific for IGF2 encoding mRNA;
   f) the analyte capture molecule for ANXA10 or encoding nucleic acid is an antibody specific for ANXA10 encoding mRNA;
   g) the analyte capture molecule for NMP-22 or encoding nucleic acid is a NMP-22 specific antibody;
   h) the analyte capture molecule for HCFHrp or encoding nucleic acid is a HCFHrp specific antibody;
   i) the analyte capture molecule for miR-200C is a single-stranded polynucleotide complementary to miR-200C;
   j) the analyte capture molecule for UPKB or encoding nucleic acid is an antibody specific for UPKB encoding mRNA;
   k) the analyte capture molecule for ABL1 or encoding nucleic acid is an antibody specific for ABL1 encoding mRNA; and
   l) the analyte capture molecule for CRH mRNA or encoding nucleic acid is an antibody specific for CRH encoding mRNA; and each sensor type is present on a different platform.
20. The multi-analyte detection system of any one of 15-19, wherein each localized surface plasmon nanostructures comprises gold.
21. The multi-analyte detection system of any one of 15-19, wherein each localized surface plasmon nanostructure comprises gold spheres.
22. The multi-analyte detection system of any one of 15-21, wherein each localized surface plasmon nanostructure is functionalized with a polyethylene glycol (PEG) chain and the analyte binding molecule is conjugated to the PEG chain.
23. A method of determining whether a human subject has bladder cancer comprising the steps of:
   a) contacting the multi-analyte detection system of any one of 15-22 with the biological sample from the subject,
   b) providing detection signal amplifiers, wherein:
      i) a first detection signal amplifier is provided to the first sensor, wherein the first detection signal amplifier comprises a BRET assembly complex, a localized surface plasmon resonance nanostructure and an analyte binding molecule for miR-205, wherein the BRET assembly complex comprises a luciferase donor conjugated to a fluorophore acceptor;
      ii) a second detection signal amplifier is provided to the second sensor, wherein the second detection signal amplifier comprises a BRET assembly complex, a localized surface plasmon resonance nanostructure and an analyte binding molecule for miR-16-1, wherein the BRET assembly complex comprises a luciferase donor conjugated to a fluorophore acceptor;
      iii) a third detection signal amplifier is provided to the third sensor, wherein the third detection signal amplifier comprises a BRET assembly complex, a localized surface plasmon resonance nanostructure and an analyte binding molecule for miR-143, wherein the BRET assembly complex comprises a luciferase donor conjugated to a fluorophore acceptor;
      iv) a fourth detection signal amplifier is provided to the fourth sensor, wherein the fourth detection signal amplifier comprises a localized surface plasmon resonance nanostructure, a BRET assembly complex and an analyte binding molecule for UCA1 nucleic acid, wherein the BRET assembly complex comprises a luciferase donor conjugated to a fluorophore acceptor;
      v) a fifth detection signal amplifier is provided to the fifth sensor, wherein the fifth sensor comprises a localized surface plasmon resonance nanostructure, a BRET assembly complex and an analyte binding molecule for IGF2 or IGF2 nucleic acid, wherein the BRET assembly complex comprises a luciferase donor conjugated to a fluorophore acceptor; and
      vi) a sixth detection signal amplifier is provided to the sixth sensor, wherein the sixth detection signal amplifier comprises a localized surface plasmon resonance nanostructure, a BRET assembly complex and an analyte binding molecule for ANXA10 or ANXA10 nucleic acid, wherein the BRET assembly complex comprises a luciferase donor conjugated to a fluorophore acceptor.
   c) adding a luciferase substrate;
   d) detecting fluorescence, localized surface plasmon resonance, bioluminescence, or surface-enhanced Raman scattering, thereby indicating the presence or amount of the analytes in the sample.
24. The method of 23, where step b further comprises:
   vii) providing a seventh signal amplifier to a seventh detection sensor, wherein the seventh detection signal amplifier comprises a localized surface plasmon resonance nanostructure, a BRET assembly complex and an analyte binding molecule for NMP-22 or NMP-22 nucleic acid, wherein the BRET assembly complex comprises a luciferase donor conjugated to a fluorophore acceptor;
   viii) providing an eighth detection signal amplifier to the eighth sensor, wherein the eighth detection signal amplifier comprises a localized surface plasmon resonance nanostructure, a BRET assembly complex and an analyte binding molecule for HCFHrp or HCFHrp nucleic acid, wherein the BRET assembly complex comprises a luciferase donor conjugated to a fluorophore acceptor;
   ix) providing a ninth detection signal amplifier to the ninth sensor wherein the ninth detection signal amplifier comprises localized surface plasmon resonance nanostructure, a BRET assembly complex and an analyte binding molecule for miR-200C nucleic acid, wherein the BRET assembly complex comprises a luciferase donor conjugated to a fluorophore acceptor;
   x) providing a tenth detection signal amplifier to the tenth sensor, wherein the tenth detection signal amplifier comprises a localized surface plasmon resonance nanostructure, a BRET assembly complex and an analyte binding molecule for UPKB or UPKB nucleic acid, wherein the BRET assembly complex comprises a luciferase donor conjugated to a fluorophore acceptor;
   xi) providing an eleventh detection signal amplifier to the eleventh sensor wherein the eleventh detection signal amplifier comprises a localized surface plasmon resonance nanostructure, a BRET assembly complex and an analyte binding molecule for ABL1 or ABL1 nucleic acid, wherein the BRET assembly complex comprises a luciferase donor conjugated to a fluorophore acceptor; and
   xii) providing a twelfth detection signal amplifier to the twelfth sensor, wherein the twelfth detection signal amplifier comprises a localized surface plasmon resonance nanostructure, a BRET assembly complex and an analyte binding molecule for CRH or CRH nucleic acid, wherein the BRET assembly complex comprises a luciferase donor conjugated to a fluorophore acceptor.
25. The method of 24, where step b further comprises providing a thirteenth detection signal amplifier to the thirteenth sensor, wherein the thirteenth detection signal amplifier comprises a localized surface plasmon resonance nanostructure, a BRET assembly complex, and an analyte binding molecule for P53 or P53 nucleic acid, wherein the BRET assembly complex comprises a luciferase donor conjugated to a fluorophore acceptor.
26. The method of 24 or 25, where step b further comprises providing a fourteenth detection signal amplifier to the fourteenth sensor, wherein the fourteenth detection signal amplifier comprises a localized surface plasmon resonance nanostructure, a BRET assembly complex, and an analyte binding molecule for KRT17or KRT17 nucleic acid, wherein the BRET assembly complex comprises a luciferase donor conjugated to a fluorophore acceptor.
27. The method of any one of 23-26, wherein
   i) the analyte binding molecule for miR-205 is a single-stranded polynucleotide complementary to miR-205;
   ii) the analyte binding molecule for miR-16-1 is a single-stranded polynucleotide complementary to miR-16-1;
   iii) the analyte binding molecule for miR-143 is a single-stranded polynucleotide complementary to miR-143;
   iv) the analyte binding molecule for UCA1 nucleic acid is a single-stranded polynucleotide complementary to UCA1 nucleic acid;
   v) the analyte binding molecule for IGF2 or an encoding nucleic acid is an antibody specific for IGF2 encoding mRNA;
   vi) the analyte binding molecule for ANXA10 or encoding nucleic acid is an antibody specific for ANXA10 encoding mRNA;
   vii) the analyte binding molecule for NMP-22 or encoding nucleic acid is a NMP-22 specific antibody;
   viii) the analyte binding molecule for HCFHrp or encoding nucleic acid is a HCFHrp specific antibody;
   ix) the analyte binding molecule for miR-200C is a single-stranded polynucleotide complementary to miR-200C;
   x) the analyte binding molecule for UPKB or encoding nucleic acid is an antibody specific for UPKB encoding mRNA;
   xi) the analyte binding molecule for ABL1 or encoding nucleic acid is an antibody specific for ABL1 encoding mRNA; and
   xii) the analyte binding molecule for CRH mRNA or encoding nucleic acid is an antibody specific for CRH encoding mRNA.
28. A method of determining whether a subject has bladder cancer comprising the steps of:
   a) contacting the multi-analyte detection system of any one of 15-22 with a biological sample from a human subject,
   b) providing detection signal amplifiers, wherein:
      i) a first detection signal amplifier is provided to the first sensor, wherein the first detection signal amplifier comprises a localized surface plasmon resonance nanostructure, a FRET assembly complex and an analyte binding molecule for miR-205, wherein the FRET assembly complex comprises a fluorophore donor conjugated to a fluorophore acceptor;
      ii) a second detection signal amplifier is provided to the second sensor, wherein the second detection signal amplifier comprises a localized surface plasmon resonance nanostructure, a FRET assembly complex and an analyte binding molecule for miR-16-1, wherein the FRET assembly complex comprises a fluorophore donor conjugated to a fluorophore acceptor;
      iii) a third detection signal amplifier is provided to the third sensor, wherein the third detection signal amplifier comprises a localized surface plasmon resonance nanostructure, a FRET assembly complex and an analyte binding molecule for miR-143 nucleic acid, wherein the FRET assembly complex comprises a fluorophore donor conjugated to a fluorophore acceptor;
      iv) a fourth detection signal amplifier is provided to the fourth sensor, wherein the fourth detection signal amplifier comprises a localized surface plasmon resonance nanostructure, a FRET assembly complex and an analyte binding molecule for UCA1 nucleic acid, wherein the FRET assembly complex comprises a fluorophore donor conjugated to a fluorophore acceptor;
      v) a fifth detection signal amplifier is provided to the fifth sensor, wherein the fifth detection signal amplifier comprises a localized surface plasmon resonance nanostructure, a FRET assembly complex and an analyte binding molecule for IGF2 or IGF2 nucleic acid, wherein the FRET assembly complex comprises a fluorophore donor conjugated to a fluorophore acceptor; and
      vi) a sixth detection signal amplifier is provided to the sixth sensor, wherein the sixth detection signal amplifier comprises a localized surface plasmon resonance nanostructure, a FRET assembly complex and an analyte binding molecule for ANXA10 or ANXA10 nucleic acid, wherein the FRET assembly complex comprises a fluorophore donor conjugated to a fluorophore acceptor;
   c) exciting each FRET assembly complex;
   d) detecting fluorescence, localized surface plasmon resonance, or surface-enhanced Raman scattering, thereby indicating the presence or amount of the analytes in the sample.
29. The method of 28, where step b further comprises:
   vii) providing a seventh detection signal amplifier to the seventh sensor, wherein the seventh detection signal amplifier comprises a localized surface plasmon resonance nanostructure, a FRET assembly complex and an analyte binding molecule for NMP-22 or NMP-22 nucleic acid, wherein the FRET assembly complex comprises a fluorophore donor conjugated to a fluorophore acceptor;
   viii) providing an eighth detection signal amplifier to the eighth sensor, wherein the eighth detection signal amplifier comprises a localized surface plasmon resonance nanostructure, a FRET assembly complex and an analyte binding molecule for HCFHrp or HCFHrp nucleic acid, wherein the FRET assembly complex comprises a fluorophore donor conjugated to a fluorophore acceptor;
   ix) providing a ninth detection signal amplifier to the ninth sensor, wherein the ninth detection signal amplifier comprises a localized surface plasmon resonance nanostructure, a FRET assembly complex and an analyte binding molecule for miR-200C nucleic acid, wherein the FRET assembly complex comprises a fluorophore donor conjugated to a fluorophore acceptor;
   x) providing a tenth detection signal amplifier to the tenth sensor, wherein the tenth detection signal amplifier comprises a localized surface plasmon resonance nanostructure, a FRET assembly complex and an analyte binding molecule for UPKB or UPKB nucleic acid, wherein the FRET assembly complex comprises a fluorophore donor conjugated to a fluorophore acceptor;
   xi) providing an eleventh detection signal amplifier to the eleventh sensor, wherein the eleventh detection signal amplifier comprises a localized surface plasmon resonance nanostructure, a FRET assembly complex and an analyte binding molecule for ABL1 or ABL1 nucleic acid, wherein the FRET assembly complex comprises a fluorophore donor conjugated to a fluorophore acceptor; and
   xii) providing a twelfth detection signal amplifier to the twelfth sensor, wherein the twelfth detection signal amplifier comprises a localized surface plasmon resonance nanostructure, a FRET assembly complex and an analyte binding molecule for CRH or CRH nucleic acid, wherein the FRET assembly complex comprises a fluorophore donor conjugated to a fluorophore acceptor.
30. The method of 29, wherein step b further comprises providing a thirteenth detection signal amplifier to the thirteenth sensor, wherein the thirteenth detection signal amplifier comprises a localized surface plasmon resonance nanostructure, a FRET assembly complex and an analyte binding molecule for P53 or P53 nucleic acid, wherein the FRET assembly complex comprises a fluorophore donor conjugated to a fluorophore acceptor.
31. The method of 29 or 30, wherein step b further comprises providing a fourteenth detection signal amplifier to the fourteenth sensor, wherein the fourteenth detection signal amplifier comprises a localized surface plasmon resonance nanostructure, a FRET assembly complex and an analyte binding molecule for KRT17or KRT17 nucleic acid, wherein the FRET assembly complex comprises a fluorophore donor conjugated to a fluorophore acceptor.
32. The method of any one of 28-31, wherein
   i) the analyte binding molecule for miR-205 is a single-stranded polynucleotide complementary to miR-205;
   ii) the analyte binding molecule for miR-16-1 is a single-stranded polynucleotide complementary to miR-16-1;
   iii) the analyte binding molecule for miR-143 is a single-stranded polynucleotide complementary to miR-143;
   iv) the analyte binding molecule for UCA1 nucleic acid is a single-stranded polynucleotide complementary to UCA1 nucleic acid;
   v) the analyte binding molecule for IGF2 or an encoding nucleic acid, is an antibody specific for IGF2 encoding mRNA;
   vi) the analyte binding molecule for ANXA10 or encoding nucleic acid is an antibody specific for ANXA10 encoding mRNA;
   vii) the analyte binding molecule for NMP-22 or encoding nucleic acid is a NMP-22 specific antibody;
   viii) the analyte binding molecule for HCFHrp or encoding nucleic acid is a HCFHrp specific antibody;
   ix) the analyte binding molecule for miR-200C is a single-stranded polynucleotide complementary to miR-200C;
   x) the analyte binding molecule for UPKB or encoding nucleic acid is an antibody specific for UPKB encoding mRNA;
   xi) the analyte binding molecule for ABL1 or encoding nucleic acid is an antibody specific for ABL1 encoding mRNA; and
   xii) the analyte binding molecule for CRH mRNA or encoding nucleic acid is an antibody specific for CRH encoding mRNA.
33. The method of 29, further comprising a thirteenth sensor, wherein the thirteenth detection signal amplifier comprises a localized surface plasmon resonance nanostructure, a FRET assembly complex and an analyte binding molecule for P53 or P53 nucleic acid, wherein the FRET assembly complex comprises a fluorophore donor conjugated to a fluorophore acceptor.
34. The method of 33 or 32, further comprises providing a fourteenth detection signal amplifier to the fourteenth sensor, wherein the fourteenth detection signal amplifier comprises a localized surface plasmon resonance nanostructure, a FRET assembly complex and an analyte binding molecule for KRT17or KRT17 nucleic acid, wherein the FRET assembly complex comprises a fluorophore donor conjugated to a fluorophore acceptor.
35. The method of any one of 23-34, wherein the sample is a urine sample.
36. The method of 35, wherein the urine sample has not undergone purification before step a.
37. A method of determining whether a human subject has bladder cancer comprising the steps of detecting the amount of miR-205, miR-16-1, miR-143, UCA1 mRNA, IGF2 or IGF2 nucleic acid, ANXA10 or ANXA10 nucleic acid in a biological sample from the subject.
38. The method of 35, further comprising detecting the amount of NMP-22 or NMP-22 nucleic acid, HCFHrp or HCFHrp nucleic acid, miR-200C, UPKB or UPKB nucleic acid, ABL1 or ABL1 nucleic acid, and CRH or CRH nucleic acid in the biological sample from the subject.
39. The method of 35 or 36, further comprising detecting the amount of P53 or P53 encoding nucleic acid in the biological sample.
40. The method of any one of 37-39, further comprising detecting the amount of KRT17or KRT17 encoding nucleic acid in the biological sample.
41. A method of treating a subject for bladder cancer comprising (a) detecting the amount of analytes associated with bladder cancer using the method any one of 23-40; and (b) administering a therapeutically effective amount of a bladder cancer therapeutic to a subject determined to have bladder cancer based in whole or in part on (a).
42. A method of treating a subject for bladder cancer comprising administering a therapeutically effective amount of a bladder cancer therapeutic to a subject determined to have bladder cancer, based in whole or in part, of the results of using the method of any one of 23-40.
43. The method of 41 or 42, wherein the bladder cancer therapeutic is selected from the group consisting of: atezolizumab, avelumab, erdafitinib, cisplatin, doxorubicin hydrochloride, enfortumab vedotin-ejfv, erdafitinib, mitomycin, pembrolizumab, nivolumab, sacituzumab govitecan-hziy, and valrubicin.
44. A method of detecting or quantifying an analyte in a sample comprising the steps of:
   a) contacting a localized surface plasmon resonance sensor with the sample, wherein the sensor comprises an analyte capture molecule;
   b) providing an analyte detection signal amplifier to the sample, wherein the analyte detection signal amplifier comprises the BRET assembly complex of 1 and binds to the analyte;
   c) adding a luciferase substrate; and
      detecting fluorescence, bioluminescence, localized surface plasmon resonance or surface-enhanced Raman scattering.
45. The method of 44, wherein said sample is a biological sample from a human patient and said method comprises using analyte detection signal amplifiers for detecting the amount of miR-205, miR-16-1, miR-143, UCA1 mRNA, IGF2 or IGF2 nucleic acid, ANXA10 or ANXA10 nucleic acid in the biological sample.
46. The method of 45, further comprising detecting the amount of NMP-22 or NMP-22 nucleic acid, HCFHrp or HCFHrp nucleic acid, miR-200C, UPKB or UPKB nucleic acid, ABL1 or ABL1 nucleic acid, or CRH or CRH nucleic acid in the biological sample.
47. The method of 44, further comprising detecting the amount of P53 or P53 encoding nucleic acid in the biological sample.
48. The method of 47, further comprising detecting the amount of KRT17or KRT17 encoding nucleic acid in the biological sample.
49. A method of treating a subject for bladder cancer comprising (a) detecting the amount of analytes associated with bladder cancer using the method any one 45-48; and (b) administering a therapeutically effective amount of a bladder cancer therapeutic to a subject determined to have bladder cancer based in whole or in part on (a).
50. A method of treating a subject for bladder cancer comprising administering a therapeutically effective amount of a bladder cancer therapeutic to a subject determined to have bladder cancer, based in whole or in part, on the results of using the method of any of 45-48.
51. The method of 49 or 50, wherein the bladder cancer therapeutic is selected from the group consisting of: atezolizumab, avelumab, erdafitinib, cisplatin, doxorubicin hydrochloride, enfortumab vedotin-ejfv, erdafitinib, mitomycin, pembrolizumab, nivolumab, sacituzumab govitecan-hziy, and valrubicin.

Examples are provided below further illustrating different features of the present invention and methodology for practicing the invention. The provided examples do not limit the claimed invention.

### EXAMPLES

### Example 1: Calibration Curve

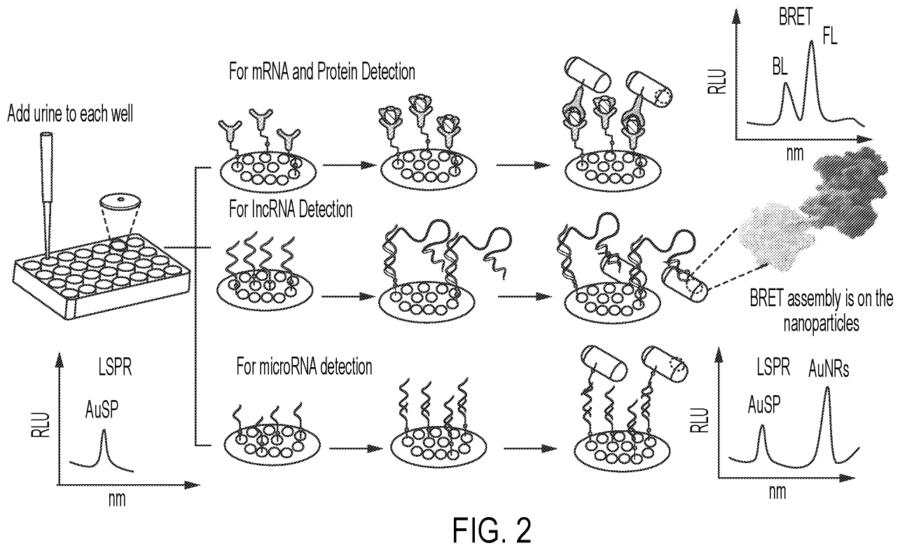

Example 1 illustrates the generation of a calibration curve using BRET assembly complexes, amplifiers targeting bladder cancer antigens, and sensors targeting bladder cancer antigens. FIG. 2 illustrates the different components and steps for the overall detections scheme.

Materials and Reagents: HAuCl₄, sodium citrate (99.5%), hexadecyltrimethylammonium bromide (CTAB, 99%), D-(+)-Glucose (99.5%), sodium borohydride, silver nitrate (99%), L (+)-ascorbic acid (reagent grade), 3-aminopropyl-triethoxysilane (APTES, 94%) were purchased from Sigma-Aldrich. Thiolated polyethylene glycols were purchased from BIOCHEMPEG. Single-stranded oligonucleotides modified 3′-SH—(CH₂)₃-ssDNA, microRNAs, and RNase H enzymes were purchased from Integrated DNA Technologies. All chemicals were used without further purifications. RNase-free sterile water and PBS buffer (pH 7.2) were obtained from Sigma-Aldrich. Corning 96-multiwell plates were purchased from Sigma-Aldrich.

Nucleic acids used as ssDNA analyte binding molecules and ssDNA analyte capture molecules are provided in the Table 4. CAP1 indicates functionalized to AuNRs and use as an analyte detection molecule. CAP2 indicates functionalized to Au spheres and use as an analyte detection molecule.

TABLE 4

| | ssDNA (oligomer) sequences | |
|---|---|---|
| Name | Sequence | Modification |
| 143-CAP1- | 5′-AGC ACT GCA CC-3′ | /3ThioMC3-D/ |
| ssDNA | SEQ ID NO: 10 | |
| 143-CAP2- | 5′- ACC AGA GAT GC -3′ | /5ThioMC6-D/ |
| ssDNA | SEQ ID NO: 11 | |
| 205-CAP1- | 5′- GGA ATG AAG GA -3′ | /3ThioMC3-D/ |
| ssDNA | SEQ ID NO: 12 | |
| 205-CAP2- | 5′- CAG ACT CCG GT -3′ | /5ThioMC6-D/ |
| ssDNA | SEQ ID NO: 13 | |
| 200C CAP1 | 5′- TGG GTA AGA CG -3′ | /3ThioMC3-D/ |
| | SEQ ID NO: 14 | |
| 200C CAP | 5′-CCA AAC ACT GC -3′ | /5ThioMC6-D/ |
| 2 | SEQ ID NO: 15 | |
| 16-1 CAP1 | 5′- ACG TGC TGC TA-3′ | /3ThioMC3-D/ |
| 5′3′ | SEQ ID NO: 16 | |
| 16-1 CAP2 | 5′-CGC CAA TAT TT -3′ | /5ThioMC6-D/ |
| 5′3′ | SEQ ID NO: 17 | |
| UCA CAP1 | 5′- CAC GGA ATG AGG GCC | /3ThioMC3-D/ |
| | AGG ACA GGG GGC CGG- 3′ | |
| | SEQ ID NO: 18 | |
| UCA CAP2 | 5′ TGA GGA CCC GAG GTC | /5ThioMC6-D/ |
| | GCA GGT GGA TCT CTT -3′ | |
| | SEQ ID NO: 19 | |

Micro RNA sequences and a UCA sequence used as analytes in generating calibration curves, are provided in Table 5.

TABLE 5

| Name | Sequence |
|---|---|
| miR-143 | 5′- GGU GCA GUG CUG CAU CUC UGG U -3′ |
| | SEQ ID NO: 20 |
| miR-205 | 5′- UCC UUC AUU CCA CCG GAG UCU G -3′ |
| | SEQ ID NO: 21 |
| miR-200c | 5′- CGU CUU ACC CAG CAG UGU UUG G -3′ |
| | SEQ ID NO: 22 |
| miR-16-1 | 5′- UAG CAG CAC GUA AAU AUU GGC G -3′ |
| | SEQ ID NO: 23 |
| UCA full | 5′- CCG GCC CCC TGT CCT GGC CCT CAT |
| length | TCC GTG AAG AGA TCC ACC TGC GAC CTC |
| | GGG TCC TCA -3′ SEQ ID NO: 24 |

Thiol-modified-ssDNAs, microRNAs, mRNAs, and proteins were kept at −20° C., and patient samples were stored at −80° C. NanoLuc®-HaloTag® Protein and Nano-Glo buffers were purchased from Promega Corporation. Washing and blocking buffers were prepared in the lab.

Analytes, the binding molecule type and the capture molecule type used in the present example are noted in Table 6. Material was obtained from commercial sources such as ProSci Inc., Novus Biological, Abbexa and Sinobiologics,

TABLE 6

| | Binding | Capture |
|---|---|---|
| Analyte | Molecule Type | Molecule Types |
| miR-205 | Polynucleotide | Polynucleotide |
| miR-16-1 | Polynucleotide | Polynucleotide |
| miR-143 | Polynucleotide | Polynucleotide |
| UCA1 nucleic acid | Polynucleotide | Polynucleotide |
| IGF2 encoding mRNA | Antibody | Antibody |
| ANXA10 encoding mRNA | Antibody | Antibody |
| NMP-22 protein | Antibody | Antibody |
| HCFHrp protein | Antibody | Antibody |
| miR-200C | Polynucleotide | Polynucleotide |
| UPKB encoding mRNA | Antibody | Antibody |
| ABL1 encoding mRNA | Antibody | Antibody |
| CRH encoding mRNA | Antibody | Antibody |

Sensor Functionalization. AuSPs incorporated into chips made up of three-dimensional nanostructures (see Example 4 infra) were functionalized using polyethylene glycol thiol (1K) with n-hydroxysuccinimide (NHS) to provide SC-PEG-S—Au. The LSPR peak of the PEG (2K)-SH functionalized AuSp chip is shown at 550 nM. Functionalization was carried out using 1 nM AuSP nanoparticles mixed with a 1:2000 mole ratio of PEG-1K at room temperature. The reaction was allowed to complete for 24 hours on a rotary shaker at medium speed. Excess PEG was removed by ultracentrifugation at 10,000 rpm for 30 minutes. The supernatant was removed and continued for three rounds of cleaning to remove all excess PEG-SH.

ssDNA loading on to Sensor Chips: Ten micromolar ssDNA disulfide was incubated in 100 μM tris(2-carboxyethyl) phosphine) (TCEP) in 1 mL 10×PBS buffer at pH 5.2, at room temperature for one hour while giving 60s sonication every 15 minutes. Sample was then diluted to the required volume to obtain 10 μM ssDNA disulfide. Sensor chips were positioned at the bottom of individual wells of a 96 white well-plate, and loaded with 200 μL 10 μM ssDNA disulfide. The plate was sealed and left in the dark, at room temperature for 12 hours. After the 12 hours, 4 μL of a 3 M NaCl solution was added five times at one-hour intervals. During each addition, the solution was sonicated for 10 seconds. Following the final sonification, wells were sealed and incubated for another 12 hours. Blocking buffer (StartingBlock™ (PBS) Blocking Buffer from Thermo fisher) was then added to the chips to avoid nonspecific binding, the functionalized chips were incubated for 30 minutes, and then chips were centered and dried at 25° C. for 2 hours before use in the analytical application.

Anti-body Loading onto Sensor Chips: Sensor chips containing C-PEG-SH (1K) functionalized AuSP were subjected to EDC/NHS coupling in MES buffer at pH 6.0 to provide COOH activation and then 20 μg/mL antibody solution in conjugate buffer solution was added. The reaction continued for eight hours, and then the chip was cleaned using a washing buffer to remove the unbound antibodies from the surface. After removing the unbound antibodies, the chip surface was blocked using a blocking buffer.

Gold Nanorods Preparation: AuNRs synthesis was carried out in two different steps. Seed solution was made by adding 250 μL of a 10 mM HAuCl₄ solution to 7.5 mL of a 100 mM CTAB solution in a 20 mL glass vial at room temperature and stirring at medium speed. After five minutes, 600 μL of 10 mM NaBH₄ solution was injected and allowed to react for 15 minutes. NaBH₄ acts as a reducing agent, and after adding NaBH₄, a yellow to light color change appears.

To prepare 250 mL of AuNR solution, 250 mL of a 0.055 M CTAB solution and 17.86 mL of a 10 mM HAuCl₄ solution were combined and mixed well. A solution of 3.57 mL of 10 mM AgNO₃ was added, followed by adding 1.96 mL of 10 mM L-ascorbic acid. The solution was mixed well. Reduction of HAuCl₄ was confirmed via the formation of a colorless solution.

Initially, 342 μL of the seed solution was injected into the AuNR solution, and the reaction was allowed to complete for at least four hours. The color changed from a colorless light wine to a brownish black, indicating the formation of AuNRs. After AuNRs formation, excess CTAB was removed by centrifuging two times. The obtained AuNRs had a wavelength of about 680 nm with an average size of ˜40 nm.

AuNR PEG Functionalization: AuNRs were redissolved in Tris buffer at pH 3.0, combined with a 1 mM/1 OD SC-PEG (1K)-SH:NH₂-PEG(1K)-SH 1:1 solution, and allowed to react for 3 hours. PEG functionalized AuNRs were extracted to chloroform using the solvent-solvent extraction method, after which the chloroform was fully evaporated.

Nucleic Acid Loading on to AuNR: Reactions were carried out in 20 mL scintillation glass vials which were cleaned using 12 M NaOH. A solution of 10 μM of ssDNA CAP2 was mixed with 100 μM TCEP solution in PBS buffer 1 mL at pH 5.2 for one hour. After 1 hour, the mixture was diluted in a PBS 10× buffer at pH 8.0 containing a 10D solution of SC-PEG-SH:NH₂-PEG-SH (1K) functionalized AuNRs. The reaction was continued for 12 hours, then 20 μL of 3 M NaCl was added five times dropwise (total addition was 1 ml) at one hour intervals. After each addition, the solution was sonicated 10s. After completion of NaCl addition, the reaction was left in a dark place for 24 hours before use.

Antibody Loading on to AuNR: SH-PEG-SC-1K and NH₂-PEG-SH functionalized AuNRs were coupled with maleimide (MA)-PEG-NH₂ in PBS buffer 10× at pH 8.0 for 4 hours. Separated AuNRs were reactivated using EDC/NHS coupling, and a 20 μg/mL antibody solution was added. The reaction continued for eight hours at room temperature in a conjugate buffer, and the AuNRs were purified via centrifugation.

ssDNA Loading on to AuNR: PEG-Lyated (SC-PEG-SH (1K):NH₂-PEG-SH (1K)) AuNRs are subjected to ligand exchange with single-stranded oligonucleotides (ssDNA). Single-stranded oligonucleotides were obtained in disulfide form, and before the ligand exchange, disulfide bonds were reduced using TCEP. Reduction was carried out on 10 M ssDNA disulfide using 100 μM TCEP in acetate buffer, 500 mM, pH 5.2 at room temperature for 1 hour and a 60s sonication every 15 minutes. After the disulfide was reduced to thiol, the solution was added to a mixture containing a 1 OD/1 mL AuNRs solution in 10 mM PBS at pH 8.0 in 12 M NaOH treated glass vials. After gentle shaking by hand, the reaction was allowed to complete for another 12 hours.

After complete reaction, a 20 μl of 3 M NaCl solution was added dropwise to each vial with gentle handshaking, followed with 10s sonication. NaCl helps reduce the repulsion forces between the two strands, increasing the ssDNA loading onto the nanoparticles. Vials were stored in a drawer (dark room temperature) for at least 24 hours before use.

BRET Assembly: Nine microliters of a 9 μg/μl solution of Nluc-HT enzyme was reacted with 0.6 μL of 1 mM Oregon green dye in the presence of 150 μL of 10% IGEPAL® CA-630, in 2 mL of 1×PBS buffer in a dark 20 mL scintillated vial. The reaction continued in a cold room on a rotating shaker at a medium speed for 16 hours. The Nluc-HT-HL-Oregon tagged molecule was directly used with no further purification.

Nluc-HT-Oregon BRET molecule preparation: After ssDNA/mRNA/protein antibodies were conjugated to AuNRs, the AuNRs were coupled with MA-PEG-NH₂ in PBS buffer 10× at pH 8.0 for 4 hours. AuNRs were separated via centrifugation, blocked on the surface using blocking buffer, and purified again via centrifugation. A 100 μL Nluc-HT-HL-Oregon BRET preassembled 1OD/mL solution was injected to AuNRs in HEPES buffer at pH 8.0, and the reaction was continued for 3 hours at 4° C. After 3 hours AuNRs coupled Nluc-HT-HL-Oregon assembly was purified using 100 MW cutoff filters.

Detection Assay: The detection assay was evaluated using different amounts of analytes added to urine. Assays were carried out using 96 well plates. Each well was prepared to detect a specific biomarker. Calibration was carried out using analytes ranging in amounts from 100 ng/ml to 100 ag/mL.

AuSP assembled wells were functionalized with either ssDNA CAP1 to capture nucleic acid or antibody to capture proteins, followed by blocking the free substrate to avoid nonspecific binding. Then 100 μL of a human urine sample, centrifuged at 3000 rpm for 15 minutes to remove all large cell debris, was mixed with 100 μL of hybridization/coupling buffer, then directly added to each well and incubated for 4 hours. Sensor capture molecules captured the specific biomarkers directly from the urine without any purification or amplification. The use of polyethylene glycol thiol as a spacer and a blocking agent eliminated unspecific molecule binding. The 96 well plate was washed with washing buffer and dried under N₂ Gas, 200 μL of blocking buffer was again added to avoid further nonspecific attachment, and let sit for 30 minutes at room temperature.

A 100 μL solution of 1 OD amplifier was added to the target well, which has already captured the target analyte. Amplifiers comprised a BRET assembly carrying AuNRs solution, having ssDNA CAP2 for nucleic acid assay or antibody for protein assay. The reaction was allowed to continue in either hybridization or coupling buffer for 4 hours. Excess AuNRs were removed, and the wells washed with washing buffer to remove all unbound AuNRs from the solution.

Finally, the well plate was placed in a GLOMAX Discover plate reader, and 100 μL of reaction buffer containing furimazine (3 μL: 10 mL) was injected into each well followed by 20s shaking of the well plate, and the generated luminescence, and fluorescence was reported.

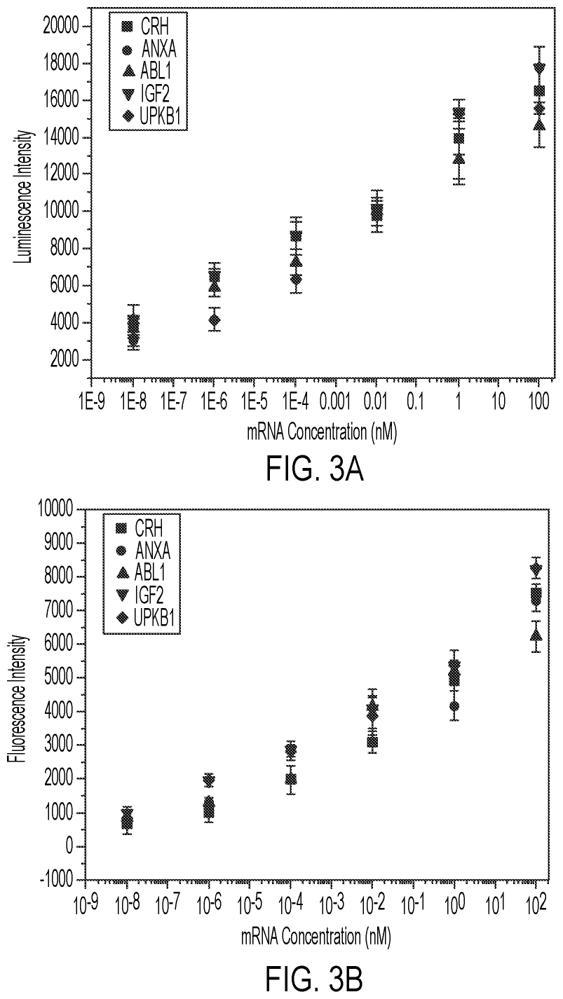

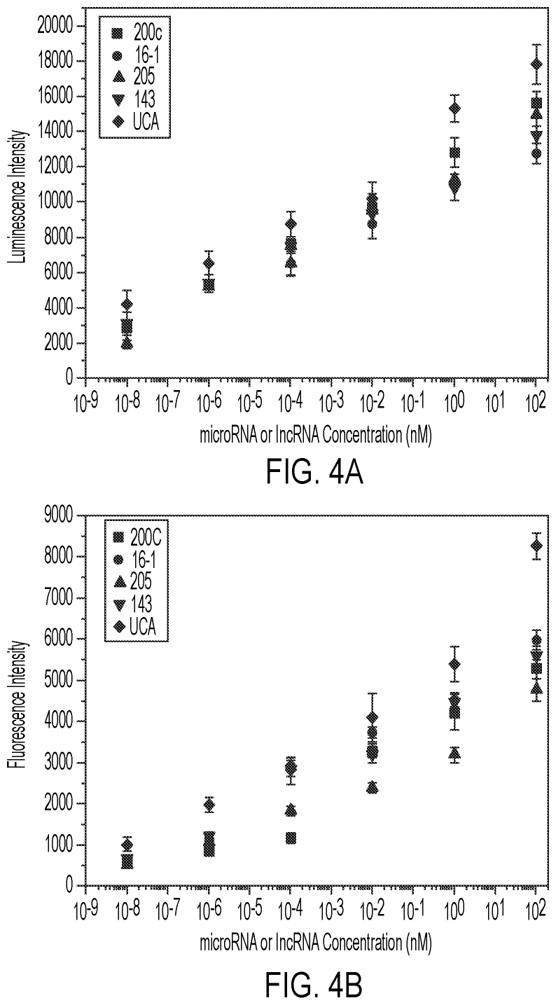

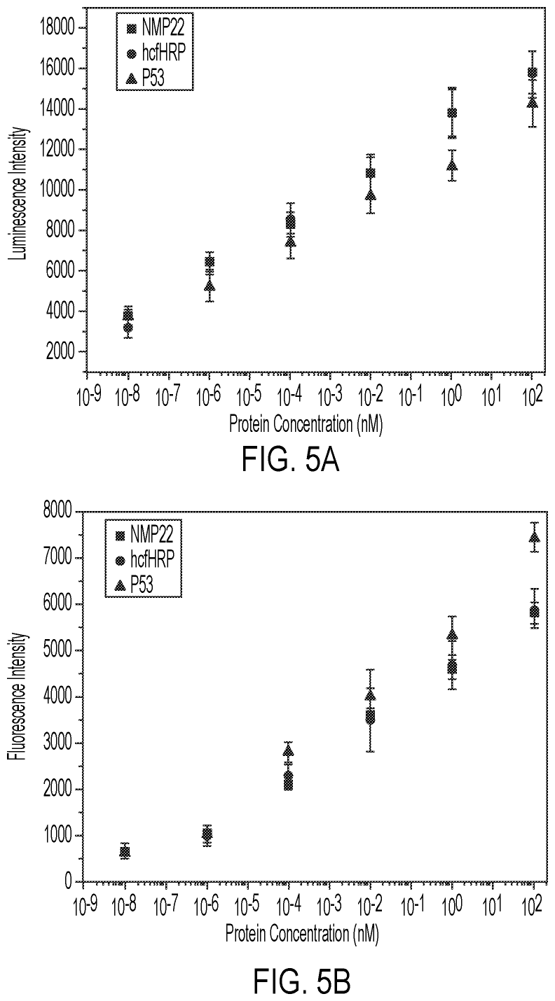

Results: Detection of analytes in the tested range of 100 ng/ml to 100 ag/ml was mostly linear. FIGS. 3A, 3B, 4A, 4B, 5A and 5B show calibration plots measuring luminescence and fluorescence for different analytes. FIG. 3A illustrates CRH, ANAX10, ABL1, IGF2 and UPKB1 luminescence. FIG. 3B illustrates CRH, ANAX10, ABL1, IGF2 and UPKB1 fluorescence. FIG. 4A illustrates miR-200c (200c), miR-16-1 (16-1), miR-205 (205), miR-143 (143) and long non-coding RNA (UCA) luminescence. FIG. 4B illustrates miR-200c (200c), miR-16-1 (16-1), miR-205 (205), miR-143 (143) and long non-coding RNA (UCA) fluorescence. FIG. 5A illustrates NMP22, HCFHrp (hcfHRP) and P53 luminescence. FIG. 5B illustrates NMP22, HCFHrp (hcfHRP) and P53 fluorescence.

Tables 7-12 provide individual data points along with the standard deviation.

TABLE 7

| Conc (nm) | CRH | std | ANXA | std | IGF2 | std | ABL1 | Std | UPKB1 | std |
|---|---|---|---|---|---|---|---|---|---|---|
| 100 | 16500 | 1240 | 15600 | 1140 | 17800 | 1184 | 14670 | 1200 | 17800 | 1120 |
| 1 | 13980 | 895 | 12800 | 1275 | 15300 | 275 | 12800 | 980 | 15300 | 775 |
| 0.01 | 10200 | 945 | 9850 | 945 | 10200 | 945 | 9800 | 850 | 10200 | 945 |
| 1.00E−04 | 8700 | 1020 | 6350 | 725 | 8750 | 720 | 7300 | 680 | 8750 | 720 |
| 1.00E−06 | 6430 | 488 | 4220 | 625 | 6520 | 688 | 5900 | 420 | 6520 | 688 |
| 1.00E−08 | 3700 | 720 | 3100 | 472 | 4200 | 820 | 3200 | 475 | 4200 | 820 |

Row intensity values obtained for mRNA luminescence assay.

TABLE 8

| Conc (nm) | CRH | std | ANXA | std | IGF2 | std | ABL1 | Std | UPKB1 | std |
|---|---|---|---|---|---|---|---|---|---|---|
| 100 | 7520 | 275 | 7300 | 320 | 8270 | 320 | 6230 | 470 | 8270 | 320 |
| 1 | 4920 | 295 | 4175 | 420 | 5400 | 420 | 5200 | 340 | 5400 | 420 |
| 0.01 | 3100 | 320 | 3900 | 580 | 4100 | 580 | 4180 | 230 | 4100 | 580 |
| 1.00E−04 | 1980 | 422 | 2800 | 225 | 2900 | 225 | 2015 | 120 | 2900 | 225 |
| 1.00E−06 | 1025 | 280 | 1285 | 180 | 1980 | 180 | 1300 | 102 | 1980 | 180 |
| 1.00E−08 | 680 | 295 | 875 | 175 | 1020 | 175 | 870 | 78 | 1020 | 175 |

Row intensity values obtained for mRNA fluorescence assay

TABLE 9

| Conc (nm) | 200C | std | 16-1 | std | 205 | std | 143 | std | UCA | std |
|---|---|---|---|---|---|---|---|---|---|---|
| 100 | 15600 | 675 | 12780 | 555 | 14920 | 565 | 13800 | 480 | 17800 | 1120 |
| 1 | 12800 | 820 | 10900 | 248 | 11300 | 290 | 10800 | 750 | 15300 | 775 |
| 0.01 | 9700 | 740 | 8720 | 752 | 9520 | 520 | 9300 | 425 | 10200 | 945 |
| 1E−4 | 7650 | 275 | 6480 | 624 | 6490 | 725 | 7500 | 275 | 8750 | 720 |
| 1E−6 | 5420 | 498 | 5240 | 325 | 5340 | 240 | 5400 | 478 | 6520 | 688 |
| 1E−8 | 3100 | 620 | 2810 | 397 | 1970 | 225 | 3200 | 590 | 4200 | 820 |

Row intensity values obtained for mRNA luminescence assay.

TABLE 10

| Conc (nm) | 200C | std | 16-1 | std | 205 | std | 143 | std | UCA | std |
|---|---|---|---|---|---|---|---|---|---|---|
| 100 | 5270 | 228 | 5980 | 225 | 4780 | 287 | 5600 | 225 | 8270 | 320 |
| 1 | 4200 | 384 | 4520 | 175 | 3200 | 184 | 4500 | 175 | 5400 | 420 |
| 0.01 | 3270 | 178 | 3740 | 125 | 2400 | 125 | 3200 | 188 | 4100 | 580 |
| 1E−4 | 1170 | 125 | 2920 | 132 | 1840 | 120 | 2800 | 320 | 2900 | 225 |
| 1E−6 | 840 | 85 | 1080 | 75 | 1070 | 105 | 1220 | 105 | 1980 | 180 |
| 1E−8 | 620 | 122 | 640 | 97 | 520 | 77 | 680 | 78 | 1020 | 175 |

Row intensity values obtained for microRNA and lncRNA fluorescence assay.

TABLE 11

| Conc (nm) | NMP22 | std | hcfHrp | std | P53 | std |
|---|---|---|---|---|---|---|
| 100 | 15800 | 1040 | 15725 | 1175 | 14285 | 1120 |
| 1 | 13800 | 1175 | 13785 | 1257 | 11200 | 775 |
| 0.01 | 10800 | 845 | 10820 | 945 | 9800 | 945 |
| 1E−4 | 8300 | 625 | 8600 | 758 | 7450 | 820 |
| 1E−6 | 6420 | 525 | 6520 | 428 | 5275 | 720 |
| 1E−8 | 3800 | 372 | 3200 | 472 | 3800 | 480 |

Row intensity values obtained for Protein luminescence assay.

TABLE 12

| Conc (nm) | NMP22 | std | hcfHrp | std | P53 | std |
|---|---|---|---|---|---|---|
| 100 | 5800 | 220 | 5900 | 420 | 7430 | 320 |
| 1 | 4575 | 230 | 4675 | 520 | 5325 | 420 |
| 0.01 | 3600 | 154 | 3500 | 680 | 4025 | 580 |
| 1E−4 | 2100 | 105 | 2275 | 275 | 2800 | 225 |
| 1E−6 | 1085 | 132 | 980 | 200 | 1055 | 180 |
| 1E−8 | 640 | 120 | 612 | 108 | 687 | 175 |

Row intensity values obtained for Protein fluorescence assay.

Table 13 summarizes equations for calibration plots for sample containing different amounts of analytes in urine. The LODs were calculated by measuring the relevant intensity for the blank sample and then calculating the Z (mean+3σ) value. The Z value was then converted into the relative concentration using the calibration curve. Blank samples were made up of nanoparticles functionalized with spacer molecule (SC-PEG(1K)-SH), but with capture molecule present. Signal amplifier solution was added and after four hours solution was removed and cleaned with washing buffer, and luminescence and fluorescence intensity were obtained by adding substrate. The first set (left side) of Table 13 provides bioluminescence, and the second set (right side) provides fluorescence.

TABLE 13

| | | | LOD | | | | LOD |
|---|---|---|---|---|---|---|---|
| Analyte | LR Equation | R² | (aM) | Analyte | LR Equation | R² | (aM) |
| CRH1 | y = 546.9ln(x) + | 0.9892 | 0.93 | CRH1 | y = 297.71ln(x) + | 0.9974 | 105.23 |
| | 13696 | | | | 5024 | | |
| ANXA | y = 587.48ln(x) + | 0.9892 | 0.47 | ANXA | y = 258.51ln(x) + | 0.9974 | 48.23 |
| | 13203 | | | | 4942.5 | | |
| ABL1 | y = 499.75ln(x) + | 0.9929 | 0.52 | ABL1 | y = 252.29ln(x) + | 0.9632 | 102.5 |
| | 12397 | | | | 5042 | | |
| IGF2 | y = 543.02ln(x) + | 0.9972 | 0.05 | IGF2 | y = 280.55ln(x) + | 0.9911 | 481 |
| | 14172 | | | | 5118 | | |
| UPKB1 | y = 545.63ln(x) + | 0.9929 | 0.01 | UPKB1 | y = 273.61ln(x) + | 0.9916 | 1126 |
| | 14127 | | | | 4942 | | |
| 200c | y = 507.75ln(x) + | 0.9984 | 2.72 | CRH1 | y = 258.51ln(x) + | 0.9974 | 69.70 |
| | 13277 | | | | 4942.5 | | |
| 16-1 | y = 447.14ln(x) + | 0.9967 | 1.13 | 16-1 | y = 264.49ln(x) + | 0.9979 | 50.59 |
| | 11264 | | | | 4822 | | |
| 205 | y = 465.32ln(x) + | 0.9954 | 0.73 | 205 | y = 247.98ln(x) + | 0.9962 | 132.78 |
| | 12528 | | | | 4606 | | |
| 143 | y = 472.14ln(x) + | 0.9979 | 0.46 | 143 | y = 235.82ln(x) + | 0.996 | 70.62 |
| | 12698 | | | | 4470 | | |
| UCA | y = 490.97ln(x) + | 0.9899 | 1.12 | UCA | y = 225.94ln(x) + | 0.9996 | 161.99 |
| | 10719 | | | | 3136.5 | | |
| NMP | y = 525.12ln(x) + | 0.9968 | 5.94 | NMP | y = 258.51ln(x) + | 0.9964 | 27.89 |
| | 13447 | | | | 4622.5 | | |
| HCFHrp | y = 537.53ln(x) + | 0.9949 | 6.38 | BTA | y = 265.79ln(x) + | 0.9997 | 740.46 |
| | 13488 | | | | 4690 | | |
| P53 | y = 528.44ln(x) + | 0.9931 | 0.01 | P53 | y = 292.61ln(x) + | 0.996 | 323.21 |
| | 13790 | | | | 5234.5 | | |

### Example 3: 3D Assembly of AuSP Particle

Different types of AuSP particles, including AuSP incorporated into a three-dimensional framework, can be produced and used as sensors. An alternative to using three dimensional AuSP particle chips, is to use a three-dimensional assembly of AuSP particles. An example of techniques that can be used to produce a three-dimensional assembly of AuSP involves activating Corning® 96-well Flat Clear glass bottom white polystyrene wells by treating with 1:1 HCl:MeOH for 1 hour. Plates are then cleaned with a copious amount of Milli-Q water and dried in a vacuum oven at 75° C. for 24 hours. The glass bottom is then treated with 300 μL of 40% N-octyl-trimethoxysilane in 200 proof ethanol for 1 hour and cleaned with ethanol 5 times to remove unbound or aggregated particles. Each time plates are sonicated for 10 minutes at a medium speed. Then, 96 well plates are dried under 75° C. for 24 hours under a vacuum oven.

N-octyl-trimethoxysilane functionalized plates are treated by adding 100 μL of SC-PEG-SH functionalized 100 nM concentration of AuSP in IPA solution to each well and letting the solution slowly evaporated for 24 hours. After 24 hours particles are washed and incubated in a running buffer for 12 hours to remove all unbound nanoparticles from the assembly.

### Example 4: Three-Dimensional Array

Example 4 illustrates techniques that can used to produce a three-dimensional array using gold nanoparticles and fiberglass. Other types of nanoparticles of different sizes and shapes, other types of fibers and alterative chemistry, as illustrated, for example, throughout the application can be employed. Nanoparticles were produced based on the procedures provided by Masterson et al., (2020) Anal. Chem. 92, 13, 9295-9304, hereby incorporated by reference here.

AuSNP-Citrate: AuSNPs (spherical) were synthesized using a published procedure (Masterson et al., (2020) Anal. Chem. 92, 13, 9295-9304) with slight modification to provide a larger volume. Briefly, a 10 ml HAuCl₄H₂O solution (4 mg/mL) was added to 390 mL H₂O in a 2 L round bottom flask on a heating mantle. The temperature was then slowly increased until the solution started to boil, then 3 mL of aqueous solution of sodium citrate (10 mg/mL) was quickly injected. The reaction was allowed to proceed for 5 minutes while stirring, and then the heat source was removed, and the red solution was allowed to cool.

AuNR-CTAB: The initial step of the AuNR (nanorod) seed solution production added 250 μL of 10 mM HAuCl₄ solution to 7.5 mL of 100 mM cetyltrimethylammonium bromide (“CTAB”) solution in a 20 mL glass vial at room temperature and stirring at medium speed. After five minutes, 600 μL of 10 mM NaBH₄ solution was injected and the reaction was continued for an additional 15 minute. NaBH₄ acts as a reducing agent, and after adding NaBH₄ a yellow to light color change appears (seed solution).

AuNR solution was prepared by adding 2 mL of 10 mM HAuCl₄ solution to 28 ml of 100 mM CTAB solution, mixing well, adding 0.4 ml of 10 mM AgNO₃, and adding 0.22 ml of 100 mM L-ascorbic acid and mixing well. The reduction of HAuCl₄ was confirmed via the formation of a colorless solution.

Finally, 48 μL of the seed solution was injected in the AuNR solution and the reaction was complete for at least four hours.

AuTNP-TEA/CTAB: AuTNP (triangles) were chemically synthesized using a previously developed procedure with minor modification. Briefly, Et₃PAu(I)Cl (18.0 mg, 0.05 mmol) was dissolved in 40 mL of CH₃CN and stirred for 10 minutes at room temperature. Next, 0.038 mL of triethylamine (“TEA”) was injected into the solution, and the temperature was gently raised. At 40° C., 600 μL of poly(methylhydrosiloxane) MW 1700 to 3200 Dalton (“PMHS”) was added, the reaction was allowed to proceed with slow magnetic stirring, and the temperature was raised to 50° C. During the reaction, the color of the solution changed from colorless to pink, purple, and light blue. The reaction stopped at a 690 nm UV peak.

Array Preparation: Three dimensional micro membranes were obtained using glass fiber sheets with or without binders and nitrocellulose sheets. Sheet parameters were basis weight 75 g/m², callaper 0.43 mm, wicking rate 5 (s/2 cm), and water absorption 79 mg/cm². The substrate was cut to the desired shape using a Carbon Steel Hollow Punch Set Kit and or custom-made dies to accommodate the well plates diameters as shown in Table 14.

TABLE 14

| Multi-Well Plates | Microwell Diameter (mm) | Cutter size (mm) |
|---|---|---|
| 96 well plates | 5 | 4.7625 mm |
| 48 well plates | 6 | 5.7625 mm |
| 24 well plates | 10, 13 | 9.7625, 12.7625 mm |
| 12 well plates | 10, 14 | 9.7625, 13.7625 mm |
| 6 well plates | 10, 14, 20 | 9.7625, 13.7625, |
| | | 19.7625 mm |

After a particular size was obtained, cutouts were washed with isopropyl alcohol and methanol, sonicated for 1 hour at a moderate speed and dried in the vacuum oven at 80° C. to remove organic solvents. This step helps remove the excess microfibers and clean the glass fiber's surface before functionalization.

The substrate was then reacted with 40% 3-aminopropyl-triethoxysilane (“APTES”) in isopropyl alcohol (“IPA”)/ethanol for 5 hours in a sealed container on a rotary shaker. Excess APTES was removed, and the substrate was cleaned in IPA/ethanol five times with 10-minute shaking intervals. After the excess APTES was removed, the fiber arrays were dried under a vacuum oven at 120° C.

Nanoparticles functionalized with Polyethylene Glycol Thiol (PEG (1k)-SH): AuSP-citrate, AuTNP-TEA/CTAB, AuNRS-CTAB, and AuNST-CTAB were separately subjected to ligand exchange with PEG-SH. Accordingly, 10 mM-PEG-SC (N-hydroxysuccinimide)-SH per 1 optical density (OD) nanoparticle solution (NPS) was dissolved in Tri-HCl buffer at pH 3.0, and the reaction was continued for 3 hours. PEG functionalized nanoparticles were separated via solvent extraction using CHCl₃.

Self-Assembly: PEGlyated nanoparticles, 20 mL of 3 OD solution, was added to 100 fiber arrays in a 50 mL centrifuge tube and placed on a rotary shaker at a medium speed for 12 hours. Chips were separated and then washed with copious amounts of chloroform. During the final wash, fiber arrays were sonicated at a medium speed in a cold bath for one hour to remove loosely bound nanoparticles. The 2D nanoparticles on the 3D fiber glass array were then ready to use.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

1. An analyte detection signal amplifier comprising (a) an analyte binding molecule; (b) a localized surface plasmon resonance nanostructure and (c) either (i) a bioluminescence resonance energy transfer (BRET) assembly complex, wherein the BRET assembly complex comprises a luciferase donor conjugated to a fluorophore acceptor or (ii) a fluorescence resonance energy transfer (FRET) assembly complex, wherein the FRET assembly complex comprises a fluorescent donor conjugated to a fluorophore acceptor.

2. The analyte detection signal amplifier of claim 1, wherein the analyte binding molecule is either a single-stranded polynucleotide, an antibody, an aptamer, a protein, a substrate, a receptor, or a ligand.

3. The analyte detection signal amplifier of claim 1, wherein the surface localized plasmon resonance nanostructure is a nanorod comprising a metal.

4. The analyte detection signal amplifier of claim 3, wherein the nanorod comprising gold.

5. The analyte detection signal amplifier of claim 1, wherein the surface localized plasmon resonance nanostructure is functionalized with polyethylene glycol (PEG), the BRET or FRET assembly complex is anchored to the localized surface plasmon resonance nanostructure by a first PEG chain, and the analyte binding molecule is anchored to the localized surface plasmon resonance nanostructure by a second PEG chain.

6. The analyte detection signal amplifier of claim 1, comprising the BRET assembly complex.

7. The analyte detection signal amplifier of claim 6, wherein the luciferase donor conjugated to the fluorophore acceptor is selected from the following combinations: NLuc-HT/HL-Oregon green, RLuc/YFP, RLuc/florescent protein (GFP), RLuc8/GFP, firefly luciferase/DsRed, RLuc/ODot, Rluc8/ODot, or Nano Luc-HT/Halotag-florescent ligand.

8. A method of detecting or quantifying an analyte in a sample comprising the steps of:
a) contacting a localized surface plasmon resonance sensor with the sample, wherein the sensor comprises an analyte capture molecule;
b) providing an analyte detection signal amplifier to the sample, wherein the analyte detection signal amplifier comprises the BRET assembly complex of claim 1 and binds to the analyte;
c) adding a luciferase substrate; and
d) detecting fluorescence, bioluminescence, localized surface plasmon resonance or surface-enhanced Raman scattering.

9. A method of detecting or quantifying an analyte in a sample comprising the steps of:
a) contacting a localized surface plasmon resonance sensor with the sample, wherein the sensor comprises an analyte capture molecule;
b) providing an analyte detection signal amplifier to the sample, wherein the analyte detection signal amplifier comprises the FRET assembly complex of claim 1, and binds to the analyte;
c) exciting the FRET assembly complex; and
d) detecting fluorescence, localized surface plasmon resonance, or surface-enhanced Raman scattering.

10. The method of claim 8, wherein the sensor comprises a metal.

11. The method of claim 10, wherein the sensor comprises gold.

12. The method of claim 8, wherein analyte signal amplifiers not bound to the analyte are removed after step b and prior to step c.

13. The method of claim 8, wherein fluorescence and/or bioluminescence is measured.

14. The method of claim 8, wherein surface-enhanced Raman scattering is measured.

15. A multi-analyte detection system comprising:
a) a first localized surface plasmon nanostructure sensor comprising an analyte capture molecule for miR-205;
b) a second localized surface plasmon nanostructure sensor comprising an analyte capture molecule for miR-16-1;
c) a third localized surface plasmon nanostructure sensor comprising an analyte capture molecule for miR-143;
d) a fourth localized surface plasmon nanostructure sensor comprising an analyte capture molecule for UCA1 nucleic acid;
e) a fifth localized surface plasmon nanostructure sensor comprising an analyte capture molecule for IGF2 or IGF2 nucleic acid; and
f) a sixth localized surface plasmon nanostructure sensor comprising an analyte capture molecule for ANXA10 or ANXA10 nucleic acid;
wherein each sensor type may be present on one or more platforms.

16. The multi-analyte detection system of claim 15, further comprising:
g) a seventh localized surface plasmon nanostructure sensor comprising an analyte capture molecule for NMP-22 or NMP-22 nucleic acid;
h) an eighth localized surface plasmon nanostructure sensor comprising an analyte capture molecule for HCFHT or HCFHip nucleic acid;
i) a ninth localized surface plasmon nanostructure sensor comprising an analyte capture molecule for miR-200C;
j) a tenth localized surface plasmon nanostructure sensor comprising an analyte capture molecule for UPKB or UPKB nucleic acid;
k) an eleventh localized surface plasmon nanostructure sensor comprising an analyte capture molecule for ABL1 or ABL1 nucleic acid; and
l) a twelfth localized surface plasmon nanostructure sensor comprising an analyte capture molecule for CRH or CRH nucleic acid; wherein each sensor type may be present on one or more platforms.

17. The multi-analyte detection system of claim 16, further comprising a localized surface plasmon nanostructure sensor comprising an analyte capture molecule for P53 or P53 nucleic acid.

18. The multi-analyte detection system of claim 16, further comprising a localized surface plasmon nanostructure sensor comprising an analyte capture molecule for KRT17or KRT17 nucleic acid.

19. The multi-analyte detection system of claim 15, wherein:
a) the analyte capture molecule for miR-205 is a single-stranded polynucleotide complementary to miR-205;
b) the analyte capture molecule for miR-16-1 is a single-stranded polynucleotide complementary to miR-16-1;
c) the analyte capture molecule for miR-143 is a single-stranded polynucleotide complementary to miR-143;
d) the analyte capture molecule for UCA1 nucleic acid is a single-stranded polynucleotide complementary to UCA1 nucleic acid;
e) the analyte capture molecule for IGF2 or an encoding nucleic acid, is an antibody specific for IGF2 encoding mRNA;
f) the analyte capture molecule for ANXA10 or encoding nucleic acid is an antibody specific for ANXA10 encoding mRNA;
g) the analyte capture molecule for NMP-22 or encoding nucleic acid is a NMP-22 specific antibody;
h) the analyte capture molecule for HCFHT or encoding nucleic acid is a HCFHip specific antibody;
i) the analyte capture molecule for miR-200C is a single-stranded polynucleotide complementary to miR-200C;
j) the analyte capture molecule for UPKB or encoding nucleic acid is an antibody specific for UPKB encoding mRNA;
k) the analyte capture molecule for ABL1 or encoding nucleic acid is an antibody specific for ABL1 encoding mRNA; and
l) the analyte capture molecule for CRH mRNA or encoding nucleic acid is an antibody specific for CRH encoding mRNA; and each sensor type is present on a different platform.

20. The multi-analyte detection system of claim 15, wherein each localized surface plasmon nanostructures comprises gold.

21. (canceled)

22. The multi-analyte detection system of claim 15, wherein each localized surface plasmon nanostructure is functionalized with a polyethylene glycol (PEG) chain and the analyte binding molecule is conjugated to the PEG chain.

23. A method of determining whether a human subject has bladder cancer comprising the steps of:
a) contacting the multi-analyte detection system of claim 15 with the biological sample from the subject,
b) providing detection signal amplifiers, wherein:
i) a first detection signal amplifier is provided to the first sensor, wherein the first detection signal amplifier comprises a BRET assembly complex, a localized surface plasmon resonance nanostructure and an analyte binding molecule for miR-205, wherein the BRET assembly complex comprises a luciferase donor conjugated to a fluorophore acceptor;
ii) a second detection signal amplifier is provided to the second sensor, wherein the second detection signal amplifier comprises a BRET assembly complex, a localized surface plasmon resonance nanostructure and an analyte binding molecule for miR-16-1, wherein the BRET assembly complex comprises a luciferase donor conjugated to a fluorophore acceptor;
iii) a third detection signal amplifier is provided to the third sensor, wherein the third detection signal amplifier comprises a BRET assembly complex, a localized surface plasmon resonance nanostructure and an analyte binding molecule for miR-143, wherein the BRET assembly complex comprises a luciferase donor conjugated to a fluorophore acceptor;
iv) a fourth detection signal amplifier is provided to the fourth sensor, wherein the fourth detection signal amplifier comprises a localized surface plasmon resonance nanostructure, a BRET assembly complex and an analyte binding molecule for UCA1 nucleic acid, wherein the BRET assembly complex comprises a luciferase donor conjugated to a fluorophore acceptor;
v) a fifth detection signal amplifier is provided to the fifth sensor, wherein the fifth sensor comprises a localized surface plasmon resonance nanostructure, a BRET assembly complex and an analyte binding molecule for IGF2 or IGF2 nucleic acid, wherein the BRET assembly complex comprises a luciferase donor conjugated to a fluorophore acceptor; and
vi) a sixth detection signal amplifier is provided to the sixth sensor, wherein the sixth detection signal amplifier comprises a localized surface plasmon resonance nanostructure, a BRET assembly complex and an analyte binding molecule for ANXA10 or ANXA10 nucleic acid, wherein the BRET assembly complex comprises a luciferase donor conjugated to a fluorophore acceptor;
c) adding a luciferase substrate; and
d) detecting fluorescence, localized surface plasmon resonance, bioluminescence, or surface-enhanced Raman scattering, thereby indicating the presence or amount of the analytes in the sample.

24. The method of claim 23, where step b further comprises:
vii) providing a seventh signal amplifier to a seventh detection sensor, wherein the seventh detection signal amplifier comprises a localized surface plasmon resonance nanostructure, a BRET assembly complex and an analyte binding molecule for NMP-22 or NMP-22 nucleic acid, wherein the BRET assembly complex comprises a luciferase donor conjugated to a fluorophore acceptor;
viii) providing an eighth detection signal amplifier to the eighth sensor, wherein the eighth detection signal amplifier comprises a localized surface plasmon resonance nanostructure, a BRET assembly complex and an analyte binding molecule for HCFHT or HCFHT nucleic acid, wherein the BRET assembly complex comprises a luciferase donor conjugated to a fluorophore acceptor;
ix) providing a ninth detection signal amplifier to the ninth sensor wherein the ninth detection signal amplifier comprises localized surface plasmon resonance nanostructure, a BRET assembly complex and an analyte binding molecule for miR-200C nucleic acid, wherein the BRET assembly complex comprises a luciferase donor conjugated to a fluorophore acceptor;
x) providing a tenth detection signal amplifier to the tenth sensor, wherein the tenth detection signal amplifier comprises a localized surface plasmon resonance nanostructure, a BRET assembly complex and an analyte binding molecule for UPKB or UPKB nucleic acid, wherein the BRET assembly complex comprises a luciferase donor conjugated to a fluorophore acceptor;
xi) providing an eleventh detection signal amplifier to the eleventh sensor wherein the eleventh detection signal amplifier comprises a localized surface plasmon resonance nanostructure, a BRET assembly complex and an analyte binding molecule for ABL1 or ABL1 nucleic acid, wherein the BRET assembly complex comprises a luciferase donor conjugated to a fluorophore acceptor; and
xii) providing a twelfth detection signal amplifier to the twelfth sensor, wherein the twelfth detection signal amplifier comprises a localized surface plasmon resonance nanostructure, a BRET assembly complex and an analyte binding molecule for CRH or CRH nucleic acid, wherein the BRET assembly complex comprises a luciferase donor conjugated to a fluorophore acceptor.

25. The method of claim 24, where step b further comprises providing a thirteenth detection signal amplifier to the thirteenth sensor, wherein the thirteenth detection signal amplifier comprises a localized surface plasmon resonance nanostructure, a BRET assembly complex, and an analyte binding molecule for P53 or P53 nucleic acid, wherein the BRET assembly complex comprises a luciferase donor conjugated to a fluorophore acceptor.

26. The method of claim 24, where step b further comprises providing a fourteenth detection signal amplifier to the fourteenth sensor, wherein the fourteenth detection signal amplifier comprises a localized surface plasmon resonance nanostructure, a BRET assembly complex, and an analyte binding molecule for KRT17or KRT17 nucleic acid, wherein the BRET assembly complex comprises a luciferase donor conjugated to a fluorophore acceptor.

27. The method of claim 23, wherein
i) the analyte binding molecule for miR-205 is a single-stranded polynucleotide complementary to miR-205;
ii) the analyte binding molecule for miR-16-1 is a single-stranded polynucleotide complementary to miR-16-1;
iii) the analyte binding molecule for miR-143 is a single-stranded polynucleotide complementary to miR-143;
iv) the analyte binding molecule for UCA1 nucleic acid is a single-stranded polynucleotide complementary to UCA1 nucleic acid;
v) the analyte binding molecule for IGF2 or an encoding nucleic acid is an antibody specific for IGF2 encoding mRNA;
vi) the analyte binding molecule for ANXA10 or encoding nucleic acid is an antibody specific for ANXA10 encoding mRNA;
vii) the analyte binding molecule for NMP-22 or encoding nucleic acid is a NMP-22 specific antibody;
viii) the analyte binding molecule for HCFHrp or encoding nucleic acid is a HCFHip specific antibody;
ix) the analyte binding molecule for miR-200C is a single-stranded polynucleotide complementary to miR-200C;
x) the analyte binding molecule for UPKB or encoding nucleic acid is an antibody specific for UPKB encoding mRNA;
xi) the analyte binding molecule for ABL1 or encoding nucleic acid is an antibody specific for ABL1 encoding mRNA; and
xii) the analyte binding molecule for CRH mRNA or encoding nucleic acid is an antibody specific for CRH encoding mRNA.

28. A method of determining whether a subject has bladder cancer comprising the steps of:
a) contacting the multi-analyte detection system of claim 15 with a biological sample from a human subject,
b) providing detection signal amplifiers, wherein:
i) a first detection signal amplifier is provided to the first sensor, wherein the first detection signal amplifier comprises a localized surface plasmon resonance nanostructure, a FRET assembly complex and an analyte binding molecule for miR-205, wherein the FRET assembly complex comprises a fluorophore donor conjugated to a fluorophore acceptor;
ii) a second detection signal amplifier is provided to the second sensor, wherein the second detection signal amplifier comprises a localized surface plasmon resonance nanostructure, a FRET assembly complex and an analyte binding molecule for miR-16-1, wherein the FRET assembly complex comprises a fluorophore donor conjugated to a fluorophore acceptor;
iii) a third detection signal amplifier is provided to the third sensor, wherein the third detection signal amplifier comprises a localized surface plasmon resonance nanostructure, a FRET assembly complex and an analyte binding molecule for miR-143 nucleic acid, wherein the FRET assembly complex comprises a fluorophore donor conjugated to a fluorophore acceptor;
iv) a fourth detection signal amplifier is provided to the fourth sensor, wherein the fourth detection signal amplifier comprises a localized surface plasmon resonance nanostructure, a FRET assembly complex and an analyte binding molecule for UCA1 nucleic acid, wherein the FRET assembly complex comprises a fluorophore donor conjugated to a fluorophore acceptor;
v) a fifth detection signal amplifier is provided to the fifth sensor, wherein the fifth detection signal amplifier comprises a localized surface plasmon resonance nanostructure, a FRET assembly complex and an analyte binding molecule for IGF2 or IGF2 nucleic acid, wherein the FRET assembly complex comprises a fluorophore donor conjugated to a fluorophore acceptor; and
vi) a sixth detection signal amplifier is provided to the sixth sensor, wherein the sixth detection signal amplifier comprises a localized surface plasmon resonance nanostructure, a FRET assembly complex and an analyte binding molecule for ANXA10 or ANXA10 nucleic acid, wherein the FRET assembly complex comprises a fluorophore donor conjugated to a fluorophore acceptor;
c) exciting each FRET assembly complex; and
d) detecting fluorescence, localized surface plasmon resonance, or surface-enhanced Raman scattering, thereby indicating the presence or amount of the analytes in the sample.

29. The method of claim 28, where step b further comprises:
vii) providing a seventh detection signal amplifier to the seventh sensor, wherein the seventh detection signal amplifier comprises a localized surface plasmon resonance nanostructure, a FRET assembly complex and an analyte binding molecule for NMP-22 or NMP-22 nucleic acid, wherein the FRET assembly complex comprises a fluorophore donor conjugated to a fluorophore acceptor;
viii) providing an eighth detection signal amplifier to the eighth sensor, wherein the eighth detection signal amplifier comprises a localized surface plasmon resonance nanostructure, a FRET assembly complex and an analyte binding molecule for HCF1lip or HCF1lip nucleic acid, wherein the FRET assembly complex comprises a fluorophore donor conjugated to a fluorophore acceptor;
ix) providing a ninth detection signal amplifier to the ninth sensor, wherein the ninth detection signal amplifier comprises a localized surface plasmon resonance nanostructure, a FRET assembly complex and an analyte binding molecule for miR-200C nucleic acid, wherein the FRET assembly complex comprises a fluorophore donor conjugated to a fluorophore acceptor;
x) providing a tenth detection signal amplifier to the tenth sensor, wherein the tenth detection signal amplifier comprises a localized surface plasmon resonance nanostructure, a FRET assembly complex and an analyte binding molecule for UPKB or UPKB nucleic acid, wherein the FRET assembly complex comprises a fluorophore donor conjugated to a fluorophore acceptor;
xi) providing an eleventh detection signal amplifier to the eleventh sensor, wherein the eleventh detection signal amplifier comprises a localized surface plasmon resonance nanostructure, a FRET assembly complex and an analyte binding molecule for ABL1 or ABL1 nucleic acid, wherein the FRET assembly complex comprises a fluorophore donor conjugated to a fluorophore acceptor; and
xii) providing a twelfth detection signal amplifier to the twelfth sensor, wherein the twelfth detection signal amplifier comprises a localized surface plasmon resonance nanostructure, a FRET assembly complex and an analyte binding molecule for CRH or CRH nucleic acid, wherein the FRET assembly complex comprises a fluorophore donor conjugated to a fluorophore acceptor.

30. The method of claim 29, wherein step b further comprises providing a thirteenth detection signal amplifier to the thirteenth sensor, wherein the thirteenth detection signal amplifier comprises a localized surface plasmon resonance nanostructure, a FRET assembly complex and an analyte binding molecule for P53 or P53 nucleic acid, wherein the FRET assembly complex comprises a fluorophore donor conjugated to a fluorophore acceptor.

31. The method of claim 29, wherein step b further comprises providing a fourteenth detection signal amplifier to the fourteenth sensor, wherein the fourteenth detection signal amplifier comprises a localized surface plasmon resonance nanostructure, a FRET assembly complex and an analyte binding molecule for KRT17 or KRT17 nucleic acid, wherein the FRET assembly complex comprises a fluorophore donor conjugated to a fluorophore acceptor.

32. The method of claim 28, wherein
i) the analyte binding molecule for miR-205 is a single-stranded polynucleotide complementary to miR-205;
ii) the analyte binding molecule for miR-16-1 is a single-stranded polynucleotide complementary to miR-16-1;
iii) the analyte binding molecule for miR-143 is a single-stranded polynucleotide complementary to miR-143;
iv) the analyte binding molecule for UCA1 nucleic acid is a single-stranded polynucleotide complementary to UCA1 nucleic acid;
v) the analyte binding molecule for IGF2 or an encoding nucleic acid, is an antibody specific for IGF2 encoding mRNA;
vi) the analyte binding molecule for ANXA10 or encoding nucleic acid is an antibody specific for ANXA10 encoding mRNA;
vii) the analyte binding molecule for NMP-22 or encoding nucleic acid is a NMP-22 specific antibody;
viii) the analyte binding molecule for HCFHrp or encoding nucleic acid is a HCFHrp specific antibody;
ix) the analyte binding molecule for miR-200C is a single-stranded polynucleotide complementary to miR-200C;
x) the analyte binding molecule for UPKB or encoding nucleic acid is an antibody specific for UPKB encoding mRNA;
xi) the analyte binding molecule for ABL1 or encoding nucleic acid is an antibody specific for ABL1 encoding mRNA; and
xii) the analyte binding molecule for CRH mRNA or encoding nucleic acid is an antibody specific for CRH encoding mRNA.

33. The method of claim 23, wherein the sample is a urine sample.

34-49. (canceled)


SEQUENCE LISTING (24 sequences)

SEQ ID NO: 1 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 2 - protein, 324 aa, Homo sapiens
MFCGDYVQGTIFPAPNFNPIMDAQMLGGALQGFDCDKDMLINILTQRCNAQRMMIAEAYQSMYGRDLIGDMREQLSDHFKDVMAGLMYPPPLYDAHELWHAMKGVGTDENCLIEILASRTNGEIFQMREAYCLQYSNNLQEDIYSETSGHFRDTLMNLVQGTREEGYTDPAMAAQDAMVLWEACQQKTGEHKTMLQMILCNKSYQQLRLVFQEFQNISGQDMVDAINECYDGYFQELLVAIVLCVRDKPAYFAYRLYSAIHDFGFHNKTVIRILIARSEIDLLTIRKRYKERYGKSLFHDIRNFASGHYKKALLAICAGDAEDY

SEQ ID NO: 3 - protein, 2115 aa, Homo sapiens
MTLHATRGAALLSWVNSLHVADPVEAVLQLQDCSIFIKIIDRIHGTEEGQQILKQPVSERLDFVCSFLQKNRKHPSSPECLVSAQKVLEGSELELAKMTMLLLYHSTMSSKSPRDWEQFEYKIQAELAVILKFVLDHEDGLNLNEDLENFLQKAPVPSTCSSTFPEELSPPSHQAKREIRFLELQKVASSSSGNNFLSGSPASPMGDILQTPQFQMRRLKKQLADERSNRDELELELAENRKLLTEKDAQIAMMQQRIDRLALLNEKQAASPLEPKELEELRDKNESLTMRLHETLKQCQDLKTEKSQMDRKINQLSEENGDLSFKLREFASHLQQLQDALNELTEEHSKATQEWLEKQAQLEKELSAALQDKKCLEEKNEILQGKLSQLEEHLSQLQDNPPQEKGEVLGDVLQLETLKQEAATLAANNTQLQARVEMLETERGQQEAKLLAERGHFEEEKQQLSSLITDLQSSISNLSQAKEELEQASQAHGARLTAQVASLTSELTTLNATIQQQDQELAGLKQQAKEKQAQLAQTLQQQEQASQGLRHQVEQLSSSLKQKEQQLKEVAEKQEATRQDHAQQLATAAEEREASLRERDAALKQLEALEKEKAAKLEILQQQLQVANEARDSAQTSVTQAQREKAELSRKVEELQACVETARQEQHEAQAQVAELELQLRSEQQKATEKERVAQEKDQLQEQLQALKESLKVTKGSLEEEKRRAADALEEQQRCISELKAETRSLVEQHKRERKELEEERAGRKGLEARLQQLGEAHQAETEVLRRELAEAMAAQHTAESECEQLVKEVAAWRERYEDSQQEEAQYGAMFQEQLMTLKEECEKARQELQEAKEKVAGIESHSELQISRQQNELAELHANLARALQQVQEKEVRAQKLADDLSTLQEKMAATSKEVARLETLVRKAGEQQETASRELVKEPARAGDRQPEWLEEQQGRQFCSTQAALQAMEREAEQMGNELERLRAALMESQGQQQEERGQQEREVARLTQERGRAQADLALEKAARAELEMRLQNALNEQRVEFATLQEALAHALTEKEGKDQELAKLRGLEAAQIKELEELRQTVKQLKEQLAKKEKEHASGSGAQSEAAGRTEPTGPKLEALRAEVSKLEQQCQKQQEQADSLERSLEAERASRAERDSALETLQGQLEEKAQELGHSQSALASAQRELAAFRTKVQDHSKAEDEWKAQVARGRQEAERKNSLISSLEEEVSILNRQVLEKEGESKELKRLVMAESEKSQKLEERLRLLQAETASNSARAAERSSALREEVQSLREEAEKQRVASENLRQELTSQAERAEELGQELKAWQEKFFQKEQALSTLQLEHTSTQALVSELLPAKHLCQQLQAEQAAAEKRHREELEQSKQAAGGLRAELLRAQRELGELIPLRQKVAEQERTAQQLRAEKASYAEQLSMLKKAHGLLAEENRGLGERANLGRQFLEVELDQAREKYVQELAAVRADAETRLAEVQREAQSTARELEVMTAKYEGAKVKVLEERQRFQEERQKLTAQVEQLEVFQREQTKQVEELSKKLADSDQASKVQQQKLKAVQAQGGESQQEAQRLQAQLNELQAQLSQKEQAAEHYKLQMEKAKTHYDAKKQQNQELQEQLRSLEQLQKENKELRAEAERLGHELQQAGLKTKEAEQTCRHLTAQVRSLEAQVAHADQQLRDLGKFQVATDALKSREPQAKPQLDLSIDSLDLSCEEGTPLSITSKLPRTQPDGTSVPGEPASPISQRLPPKVESLESLYFTPIPARSQAPLESSLDSLGDVFLDSGRKTRSARRRTTQIINITMTKKLDVEEPDSANSSFYSTRSAPASQASLRATSSTQSLARLGSPDYGNSALLSLPGYRPTTRSSARRSQAGVSSGAPPGRNSFYMGTCQDEPEQLDDWNRIAELQQRNRVCPPHLKTCYPLESRPSLSLGTITDEEMKTGDPQETLRRASMQPIQIAEGTGITTRQQRKRVSLEPHQGPGTPESKKATSCFPRPMTPRDRHEGRKQSTTEAQKKAAPASTKQADRRQSMAFSILNTPKKLGNSLLRRGASKKALSKASPNTRSGTRRSPRIATTTASAATAAAIGATPRAKGKAKH

SEQ ID NO: 4 - protein, 270 aa, Homo sp.
MWLLVSVILISRISSVGGEAMFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRSTISAEKCGPPPPIDNGDITSFLLSVYAPGSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCEEK

SEQ ID NO: 5 - protein, 260 aa, Homo sapiens
MAKDNSTVRCFQGLLIFGNVIIGCCGIALTAECIFFVSDQHSLYPLLEATDNDDIYGAAWIGIFVGICLFCLSVLGIVGIMKSSRKILLAYFILMFIVYAFEVASCITAATQQDFFTPNLFLKQMLERYQNNSPPNNDDQWKNNGVTKTWDRLMLQDNCCGVNGPSDWQKYTSAFRTENNDADYPWPRQCCVMNNLKEPLNLEACKLGVPGFYHNQGCYELISGPMNRHAWGVAWFGFAILCWTFWVLLGTMFYWSRIEY

SEQ ID NO: 6 - protein, 1130 aa, Homo sp.
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 7 - protein, 444 aa, Homo sapiens
MGGHPQLRLVKALLLLGLNPVSASLQDQHCESLSLASNISGLQCNASVDLIGTCWPRSPAGQLVVRPCPAFFYGVRYNTTNNGYRECLANGSWAARVNYSECQEILNEEKKSKVHYHVAVIINYLGHCISLVALLVAFVLFLRLRPGCTHWGDQADGALEVGAPWSGAPFQVRRSIRCLRNIIHWNLISAFILRNATWFVVQLTMSPEVHQSNVGWCRLVTAAYNYFHVTNFFWMFGEGCYLHTAIVLTYSTDRLRKWMFICIGWGVPFPIIVAWAIGKLYYDNEKCWFGKRPGVYTDYIYQGPMILVLLINFIFLFNIVRILMTKLRASTTSETIQYRKAVKATLVLLPLLGITYMLFFVNPGEDEVSRVVFIYFNSFLESFQGFFVSVFYCFLNSEVRSAIRKRWHRWQDKHSIRARVARAMSIPTSPTRVSFHSIKQSTAV

SEQ ID NO: 8 - protein, 393 aa, Homo sapiens [sequence omitted]

SEQ ID NO: 9 - protein, 432 aa, Homo sapiens
MTTSIRQFTSSSSIKGSSGLGGGSSRTSCRLSGGLGAGSCRLGSAGGLGSTLGGSSYSSCYSFGSGGGYGSSFGGVDGLLAGGEKATMQNLNDRLASYLDKVRALEEANTELEVKIRDWYQRQAPGPARDYSQYYRTIEELQNKILTATVDNANILLQIDNARLAADDFRTKFETEQALRLSVEADINGLRRVLDELTLARADLEMQIENLKEELAYLKKNHEEEMNALRGQVGGEINVEMDAAPGVDLSRILNEMRDQYEKMAEKNRKDAEDWFFSKTEELNREVATNSELVQSGKSEISELRRTMQALEIELQSQLSMKASLEGNLAETENRYCVQLSQIQGLIGSVEEQLAQLRCEMEQQNQEYKILLDVKTRLEQEIATYRRLLEGEDAHLTQYKKEPVTTRQVRTIVEEVQDGKVISSREQVHQTTR

SEQ ID NO: 10 - DNA, 11 bp, synthetic construct
  source 1..11: mol_type = other DNA
agcactgcacc

SEQ ID NO: 11 - DNA, 11 bp, synthetic construct
  source 1..11: mol_type = other DNA
accagagatgc

SEQ ID NO: 12 - DNA, 11 bp, synthetic construct
  source 1..11: mol_type = other DNA
ggaatgaagga

SEQ ID NO: 13 - DNA, 11 bp, synthetic construct
  source 1..11: mol_type = other DNA
cagactccggt

SEQ ID NO: 14 - DNA, 11 bp, synthetic construct
  source 1..11: mol_type = other DNA
tgggtaagacg

SEQ ID NO: 15 - DNA, 11 bp, synthetic construct
  source 1..11: mol_type = other DNA
ccaaacactgc

SEQ ID NO: 16 - DNA, 11 bp, synthetic construct
  source 1..11: mol_type = other DNA
acgtgctgcta

SEQ ID NO: 17 - DNA, 11 bp, synthetic construct
  source 1..11: mol_type = other DNA
cgccaatattt

SEQ ID NO: 18 - DNA, 30 bp, synthetic construct
  source 1..30: mol_type = other DNA
cacggaatgagggccaggacagggggccgg

SEQ ID NO: 19 - DNA, 30 bp, synthetic construct
  source 1..30: mol_type = other DNA
tgaggacccgaggtcgcaggtggatctctt

SEQ ID NO: 20 - RNA, 22 nt, Homo sapiens
  source 1..22: mol_type = other RNA
ggtgcagtgctgcatctctggt

SEQ ID NO: 21 - RNA, 22 nt, Homo sapiens
  source 1..22: mol_type = other RNA
tccttcattccaccggagtctg

SEQ ID NO: 22 - RNA, 22 nt, Homo sapiens
  source 1..22: mol_type = other RNA
cgtcttacccagcagtgtttgg

SEQ ID NO: 23 - RNA, 22 nt, Homo sapiens
  source 1..22: mol_type = other RNA
tagcagcacgtaaatattggcg

SEQ ID NO: 24 - DNA, 60 bp, synthetic construct
  source 1..60: mol_type = other DNA
ccggccccctgtcctggccctcattccgtgaagagatccacctgcgacctcgggtcctca